SEQUENCE LISTING (23 sequences)

SEQ ID NO: 1 - DNA, 35 bp, Artificial Sequence
  Primer
ctcaccgccattcatgaaggggtggagcctgcctg

SEQ ID NO: 2 - DNA, 35 bp, Artificial Sequence
  Primer
ctcaccgccgcccatgaaggggtggagcctgcctg

SEQ ID NO: 3 - DNA, 20 bp, Artificial Sequence
  Primer
aggcctcgacgccctgggtc

SEQ ID NO: 4 - DNA, 23 bp, Artificial Sequence
  Primer
tcagccggactgtgcactgcggc

SEQ ID NO: 5 - DNA, 22 bp, Artificial Sequence
  Primer
agccctgccacaggcttctgtg

SEQ ID NO: 6 - DNA, 23 bp, Artificial Sequence
  Primer
agtgcttatgcctgaggaatctg

SEQ ID NO: 7 - DNA, 22 bp, Artificial Sequence
  Primer
tctacagagacgtaggctgtca

SEQ ID NO: 8 - DNA, 20 bp, Artificial Sequence
  Primer
cttgagcacgagcttggtag

SEQ ID NO: 9 - DNA, 22 bp, Artificial Sequence
  Primer
tctacagagacgtaggctgtca

SEQ ID NO: 10 - DNA, 20 bp, Artificial Sequence
  Primer
cttgagcacgagcttggtag

SEQ ID NO: 11 - DNA, 20 bp, Artificial Sequence
  Primer
ccagagtccagctcatatcg

SEQ ID NO: 12 - DNA, 22 bp, Artificial Sequence
  Primer
ctcacaggtaagtggagaatgg

SEQ ID NO: 13 - DNA, 21 bp, Artificial Sequence
  Primer
gtgcattaagtggtctttatt

SEQ ID NO: 14 - DNA, 23 bp, Artificial Sequence
  Primer
tgtggtttaatagactattccta

SEQ ID NO: 15 - DNA, 20 bp, Artificial Sequence
  Primer
accacagtccatgccatcac

SEQ ID NO: 16 - DNA, 21 bp, Artificial Sequence
  Primer
tccaccaccctggttgctgta

SEQ ID NO: 17 - DNA, 25 bp, Artificial Sequence
  Primer
catggctgagggctccgccgccacg

SEQ ID NO: 18 - DNA, 25 bp, Artificial Sequence
  Primer
gggtctcctttacccatgttgacag

SEQ ID NO: 19 - DNA, 25 bp, Artificial Sequence
  Primer
gcgagcttcaccatgatggcggcgg

SEQ ID NO: 20 - DNA, 25 bp, Artificial Sequence
  Primer
cctcttacctcagttacaatttata

SEQ ID NO: 21 - DNA, 27 bp, Artificial Sequence
  Primer
ccattcatgaaggggtggagcctgcct

SEQ ID NO: 22 - protein, 5 aa, Artificial Sequence
  Recognition sequence
CCATN

SEQ ID NO: 23 - DNA, 27 bp, Artificial Sequence
  Primer
aggcaggctccaccccttcatgaatgg

(19) United States
(12) Patent Application Publication
(10) Pub. No.: US 2005/0054012 A1
(43) Pub. Date: Mar. 10, 2005

(54) Methods of detecting and treating facioscapulohumeral muscular dystrophy

(76) Inventors: Rossella G. Tupler, Auburn, MA (US); Michael R. Green, Boylston, MA (US); Davide Gabellini, Worcester, MA (US)

Correspondence Address: FISH & RICHARDSON PC, 225 FRANKLIN ST, BOSTON, MA, 02110, US

(21) Appl. No.: 10/686,491

(22) Filed: Oct. 14, 2003

Related U.S. Application Data
(60) Provisional application No. 60/418,024, filed on Oct. 11, 2002.

(51) Int. Cl. G01N 33/53; G01N 33/567

(52) U.S. Cl. 435/7.2

(57) ABSTRACT

The present invention includes methods and compositions useful for treating or preventing facioscapulohumeral muscular dystrophy (FSHD), and animal models useful for identifying compounds effective in treating or preventing FSHD.

### CLAIM OF PRIORITY

This application claims priority under 35 USC §119(e) to U.S. Provisional Patent Application Ser. No. 60/418,024, filed on Oct. 11, 2002, the entire contents of which are hereby incorporated by reference.

### FEDERALLY SPONSORED RESEARCH OR DEVELOPMENT

This invention was made with Government support under Grants Nos. R21 NS43973 and RO1 NS047584-01 awarded by the National Institutes of Health. The U.S. Government has certain rights in the invention.

### TECHNICAL FIELD

This application relates to muscular dystrophy.

### BACKGROUND

Facioscapulohumeral muscular dystrophy (FSHD) is the third most common inherited myopathy, with an incidence of 1:20,000 (Padberg, 1982). The disease is characterized by progressive weakness and atrophy of the facial and shoulder girdle muscles, which subsequently spreads to the abdominal and pelvic girdle muscles with highly variable expression. The genetic defect follows autosomal dominant inheritance and new mutations account for approximately 10% of recognized cases (Lunt, 1998).

The FSHD locus was mapped to the subtelomeric region of the long arm of chromosome 4, 4q35, by genetic linkage analysis (Sarfarazi et al., 1992). Most FSHD patients carry rearrangements occurring in a 3.3 kilobase (kb) tandemly repeated sequence (D4Z4) located at the 4q subtelomeric region (Wijmenga et al., 1992). These rearrangements result in EcoRI alleles that are shorter than 35 kb. This feature provides a molecular marker for FSHD diagnosis (Lunt, 1998).

D4Z4 is highly polymorphic with a variable number tandem repeat (VNTR) structure (Hewitt et al., 1994; Winokur et al., 1994). In the general population, D4Z4 can vary between about 11 and 150 units, whereas FSHD patients carry fewer than 11 repeats (Lunt, 1998). The number of D4Z4 repeats is a critical determinant of the age of onset and clinical severity of FSHD (Goto et al., 1995; Lunt et al., 1995; Zatz et al., 1995; Tawil et al., 1996; Hsu et al., 1997; Ricci et al., 1999). In general, 1-3 D4Z4 repeats is associated with a severe form of disease that presents in childhood, 4-7 repeats are present in the most common form of FSHD, and 8-10 repeats are associated with milder disease and reduced penetrance.

Some normal individuals carry an abnormal chromosome 4 resulting from an unbalanced translocation between the 4q35 subtelomeric region and the short arm of an acrocentric chromosome 4 (Tupler et al., 1996). The rearranged chromosome 4 lacks the entire D4Z4 repeat and a 200 kb proximal region including FRG2, TUB4q, and FRG1. Thus, haploinsufficiency of the entire 4q subtelomeric region has no phenotypic consequence, whereas deletion of only the D4Z4 repeats is associated with FSHD. Collectively, these results suggest that FSHD results from a gain-of-function mutation and raise the possibility that 4q35 genes proximal to D4Z4 play a role in disease initiation or progression.

### SUMMARY

The present invention is based on the discovery that overexpression of the 4q35 genes plays a role in the etiology of facioscapulohumeral muscular dystrophy (FSHD), and that these genes, when overexpressed in a transgenic animal, provide an animal model of muscular dystrophy. Thus, the invention includes methods and compositions useful for treating or preventing FSHD, and animal models useful for identifying compounds effective in treating or preventing FSHD.

In one aspect, the invention relates to transgenic animals, e.g., non-human mammals, overexpressing a 4q35 gene. The 4g35 gene can be FSHD region gene 1 (FRG1), FSHD region gene 2 (FRG2), or adenine nucleotide translocator-1 gene (ANT1). In some embodiments, the transgenic animal overexpresses a 4q35 gene in some of its cells, e.g., is heterozygous. In other embodiments, the transgenic animal overexpresses one or more 4q35 genes in all of its cells, e.g., is homozygous. In some embodiments, the overexpression is due to additional copies of a 4q35 gene; in other embodiments, the overexpression is due to the presence of a promoter that allows increased expression of a 4q35 gene. In some embodiments, the promoter is an inducible promoter. In some embodiments, the 4q35 gene is overexpressed in a tissue-specific manner, e.g., only overexpressed in muscle cells.

In one aspect, the invention relates to a method of identifying a candidate compound for treating FSHD. The method includes the steps of providing a D4Z4 binding element, e.g., DNA comprising one or more D4Z4 repeats, contacting the D4Z4 binding element with a test compound, and determining whether the test compound interacts with the D4Z4 binding element, such that an interaction between the D4Z4 binding element and the test compound indicates that the test compound is a candidate compound. In some embodiments, the D4Z4 binding element is in a cell that expresses a 4q35 gene. In some embodiments, the interaction is binding of the test compound to the D4Z4 binding element. In some embodiments, the interaction is the disruption of the binding of a D4Z4-binding protein to the D4Z4 binding element. The method can include the step of determining the level of expression of a 4q35 gene compared to a reference (such as an assay mixture that is not contacted with the test compound). The 4q35 gene can be FSHD region gene 1 (FRG1), FSHD region gene 2 (FRG2), or adenine nucleotide translocator-1 gene (ANT1). The cell can be, for example, a muscle cell (e.g., from a subject that has FSHD, or from a transgenic animal as described herein).

In another aspect, the invention includes a method of identifying a candidate compound for treating FSHD. The method includes the steps of providing a cell that can express a D4Z4 recognition complex (DRC) component, contacting the cell with a test compound, measuring expression of the DRC component, such that an increase in expression of the DRC component compared to a reference cell that was not contacted with the test compound indicates that the test compound is a candidate compound for treating FSHD. The DRC component can be YY1, HMGB2, or nucleolin.

In some embodiments, the invention is a method of identifying a candidate compound for treating FSHD. The invention includes the steps of providing an D4Z4 binding element and a DRC such that they can interact, contacting the D4Z4 binding element and DRC or a DRC component with a test compound, and determining whether the test compound affects the interaction between the D4Z4 binding element and the DRC or DRC component, such that an increase in the interaction between the D4Z4 binding element and the DRC or DRC component in the presence of the test compound indicates that the test compound is a candidate compound. The DRC component can be YY1, HMGB2, or nucleolin.

In another aspect the invention is a method of determining whether a treatment for FSHD is effective. The method includes the steps of obtaining a biological sample from a subject being treated for FSHD, determining the level of expression of a 4q35 gene, comparing the level of expression of the 4q35 gene to a reference, such that a decrease in the level of expression of the FSHD gene relative to a reference indicates that the FSHD treatment is effective. Alternatively, the method includes administering the treatment to a transgenic animal model of FSHD, and determining whether the treatment is effective to treat or prevent the disease, e.g., prevent the onset of symptoms, reduce the severity of symptoms, or reverse the symptoms of the disease.

The 4q35 gene can be FSHD region gene 1 (FRG1), FSHD region gene 2 (FRG2), or adenine nucleotide translocator-1 gene (ANT1). In some embodiments, expression of at least two 4q35 genes is decreased in the biological sample.

The invention includes a method of treating a subject having or at risk for FSHD that includes the steps of administering to the subject a compound that increases the expression or activity of at least one component of a D4Z4 recognition complex (DRC). The component of the D4Z4 recognition complex can be YY1, HMGB2, or nucleolin.

In another embodiment, the invention relates to a method of identifying a subject having or at risk of having FSHD that includes the steps of obtaining a biological sample from the subject, determining the level of expression of a 4q35 gene in the sample, comparing the level of expression of the 4q35 gene in the sample compared to a control obtained from an individual that does not have FSHD, such that increased expression of the 4q35 gene indicates that the subject is at risk of having or has FSHD. The 4q35 gene can be FSHD region gene 1 (FRG1), FSHD region gene 2 (FRG2), or adenine nucleotide translocator-1 gene (ANT1). The biological sample can be, e.g., blood or a muscle biopsy.

Candidate compounds identified as described herein can be used to treat a subject having or at risk for FSHD by administering a therapeutically effective amount of the compound to the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIG. 1A is a pair of bar graphs showing the expression level of FRG2, GRG1, and ANT1 in normal muscle (top graph) and as a relative expression level in FSHD muscle compared to normal muscle (bottom graph), as determined using RT-PCR analysis.

FIG. 1B is a schematic representation of 4q35, showing physical distances between the genes in megabases (Mb).

FIGS. 1C and 1D are representations of phosphoimager analysis showing the expression level of FRG2, FRG1, ANT1, and GAPH in RNA extracted from peripheral blood lymphocytes (PBL) of the same normal and FSHD patients as in (FIG. 1A) by radioactive RT-PCR (1C) and muscle from two other normal individuals and patients affected by Duchenne Muscular Dystrophy (DMD), Amyotrophic Lateral Sclerosis (ALS) and Limb Girdle Muscular Dystrophy type 2 (LGMD) patients (FIG. 1D).

FIGS. 1E and 1F are a representation of a phosphoimager analysis (1E) of FRG2 and GAPDH expression in muscle from two additional normal (Normal 6 and 7) and six additional FSHD patients (FSHD 4-9) carrying the indicated number of D4Z4 repeats. The bar graph (1F) shows the results of analysis of the phosphoimager analysis in which signal intensity was quantitated by phosphoimager analysis and normalized to the GAPDH signal. The resulting ratios of expression levels between FSHD muscles and normal muscle are shown.

FIG. 2A is an autoradiogram of an electrophoretic mobility shift assay (EMSA) showing binding of nuclear extract to various KpnI fragments from D4Z4. A schematic drawing of the DZ4Z region is below the autoradiogram and illustrates the positions of the KpnI fragments and the length of each fragment, and the positions of KpnI (K), PstI (P), and Eagl (E) sites within the fragment.

FIG. 2B is a representation of a phosphorimage showing the results of experiments in which the D4Z4 probe was incubated with (+) or without (−) nuclear extract, in the presence of a molar excess of cold specific or nonspecific competitor as indicated. The mobility shift is indicated by the arrowhead and the position of the free probe is indicated by the bracket.

FIG. 2C is a representation of an autoradiogram showing the results of an in vitro DNase I footprinting assay in which D4Z4 was end-labeled and incubated with our without DNase I for 1 minute in the presence or absence of increasing quantities of either HeLa, C2C12 myoblast, or C2C12 myotube nuclear extract. The black bar represents the protected region, and the corresponding 27 bp sequence is indicated. AG+A sequencing ladder (not shown) was run in parallel with the footprinting reactions to orient the sequence.

FIG. 3A is a schematic representation of the reporter constructs containing the CMV promoter driving expression of the neomycin resistance reporter gene (NEO). One, two, or six copies of the 27 bp minimal D4Z4 binding element (DBE) were inserted between the CMV promoter and the transcription start site of the NEO gene which is indicated by the arrow. A construct containing a pBluescript spacer equivalent in length to the size of the insert generated by six copies of the DBE was also included as a control.

FIG. 3B is a bar graph showing the results of experiments in which HeLa cells were stably transfected and grown in medium containing G418. G418-resistant colonies were counted after two weeks; the number of colonies obtained with each construct is shown relative to the number of colonies obtained for the construct lacking D4z4 binding sites.

FIGS. 4A and 4B are reproductions of gels showing the results of analysis of human FRG1 gene expression performed on RNA extracted from different tissues (soleus and paravertebral skeletal muscles, brain, liver, kidney and heart) derived from one normal mouse (wild type) and two HSA-FRG1 transgenic mice (FRG1 TgF19 and FRG1 TgF42) by RT-PCR using specific primers (4A). GAPDH expression was also monitored and used for sample normalization (4B). The HSA-FRG1 transgene is expressed uniquely in HSA-FRG1 transgenic skeletal muscles.

FIG. 4C is a reproduction of an immunoblot performed on total tissue homogenates to confirm the specific expression of FRG1 protein in muscle tissues using FRG1 specific antibodies (upper panel). Coomassie™ staining is shown as a loading reference (lower panel). FRG1 protein is expressed exclusively in the in HSA-FRG1 transgenic skeletal muscles.

FIGS. 4D and 4E are photographs showing the phenotype of the HSA-FRG1 transgenic mouse. Gross appearances of three-month-old mice are shown. Marked reduction of body size and spine deformity are visible in the HSH FRG1 mouse (4E). A wild type mouse is shown in FIG. 4D. The “hunchback” phenotype is present in all HSA-FRG1 transgenic mouse strains.

FIGS. 5A and 5B are photomicrographs showing sections from tibialis anterior, revealing muscle fibers of various caliber and many muscle fibers with central nuclei (arrows) that are typical of a dystrophic process.

FIGS. 6A and 6B are photomicrographs showing sections illustrating the cellular changes present in the diaphragm of normal (6A) and HAS-FRG1 (6B) mice.

FIGS. 7A and B are photomicrographs showing sections of soleus muscle: the architecture is almost intact with very few cell with central nuclei; the muscle weight in the HAS-FRG1 mouse (7B) is comparable to the normal control (7A).

FIGS. 8A-8D are photomicrographs showing fiber-type composition in muscles obtained from normal (FIGS. 8A-B) and HSA-FRG1 transgenic animals (FIGS. 8C-8D).

FIG. 9 is a schematic diagram illustrating the generation of transgenic animals overexpressing FRG1, FRG2, or ANT1. The top panel is a schematic illustration of an exemplary construct suitable for use in the methods described herein.

FIG. 10 is a schematic diagram illustrating a 4q35 gene overexpression model of the autosomal dominant transmission of FSHD.

FIG. 11A is a schematic representation of the chromatographic steps used to purify the D4Z4 binding activity. HeLa nuclear extracts were fractionated first on a P11 Phosphocellulose column, and the active fraction, as monitored by EMSA, was subsequently fractionated on a DEAE-Sepharose column. Active fractions (bracketed lanes) were pooled and loaded onto a DBE affinity resin; the active fractions from this step were pooled and fractionated again on a fresh DBE affinity column. Final active fractions (bracketed lanes) were pooled and separated by SDS-PAGE, and four bands were detected by silver staining (left). Mass spectrometry analysis identified the bands as nucleolin, YY1, keratin, and HMGB2, as shown. The mass spectrometry results were confirmed by immunoblotting (right).

FIG. 11B shows the results of EMSA performed in the presence of antibodies specific to each DRC component, or in the presence of the control antibody IgG Anti-YY1 antibodies immuno-competed the mobility shift, while anti-Nucleolin and anti-HMGB2 antibodies generated a supershift (indicated by the arrow).

FIG. 11C shows the results of EMSA performed with purified GST-YY1 (left) or nuclear extract (right) using a wildtype (wt) DBE probe or a version mutated in the YY1 recognition sequence, as shown.

FIG. 12A is a reproduction of an immunoblot showing the result of chromatin immunoprecipitation (ChIP) analysis of D4Z4 binding in HeLa cells.

FIG. 12B is a reproduction of an immunoblot showing the result of ChIP analysis in a human/rodent monochromosomal cell hybrid containing a single human chromosome 4 such that the only genomic copy of D4Z4 is present at human 4q35. Samples were treated and analyzed as described in (A).

FIG. 12C is a schematic representation of the region analyzed in 12A-B showing the relative positions of D4Z4, p13E-11, and FRG1.

FIG. 13A is an immunoblot of HeLa cells transfected with morpholino antisense oligonucleotides specific for YY1, HMGB2, or nucleolin or with a control morphlino oligonucleotide.

FIG. 13B is an immunoblot of FRG2 and GAPDH expression analysis performed by RTPCR analysis on RNA extracted from the transfected cells shown in 13A.

### DETAILED DESCRIPTION

FSHD is a complex disease that involves a specific set of muscle groups. The disease is highly variable in its severity, and progression of the disease is unpredictable. The disease has been causally related to deletion of subtelomeric D4Z4 repeats at 4q35; however, no candidate gene has been isolated.

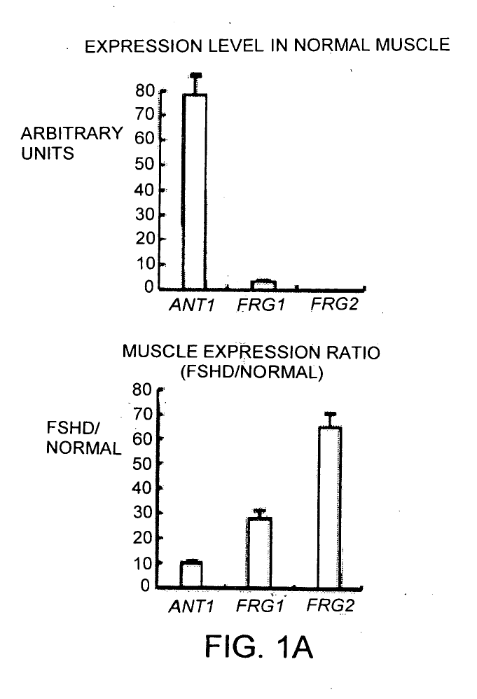

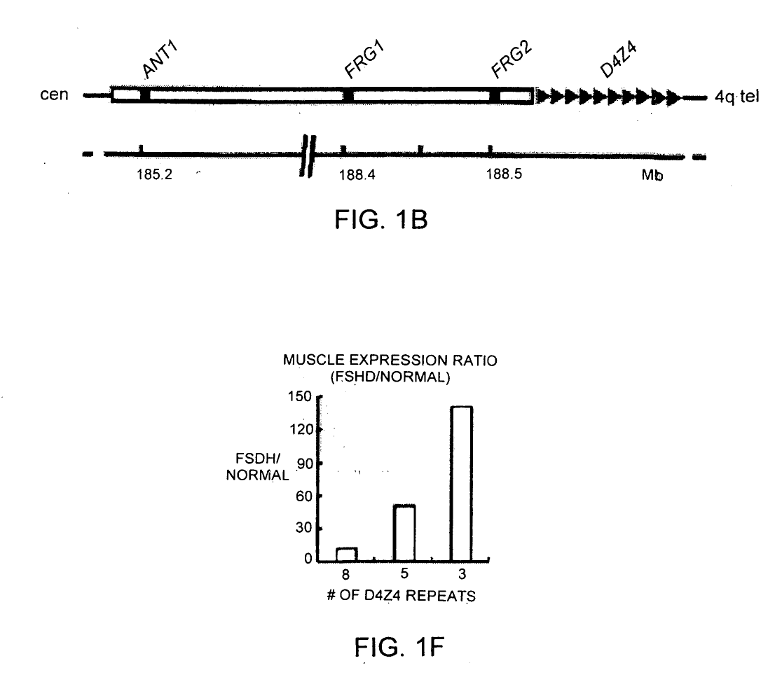

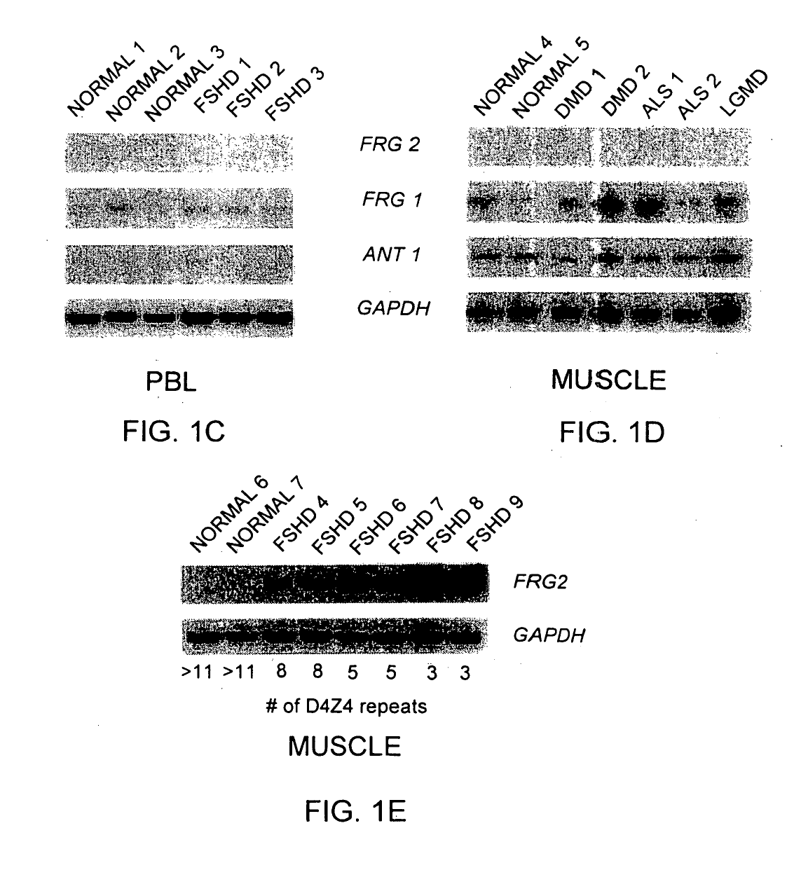

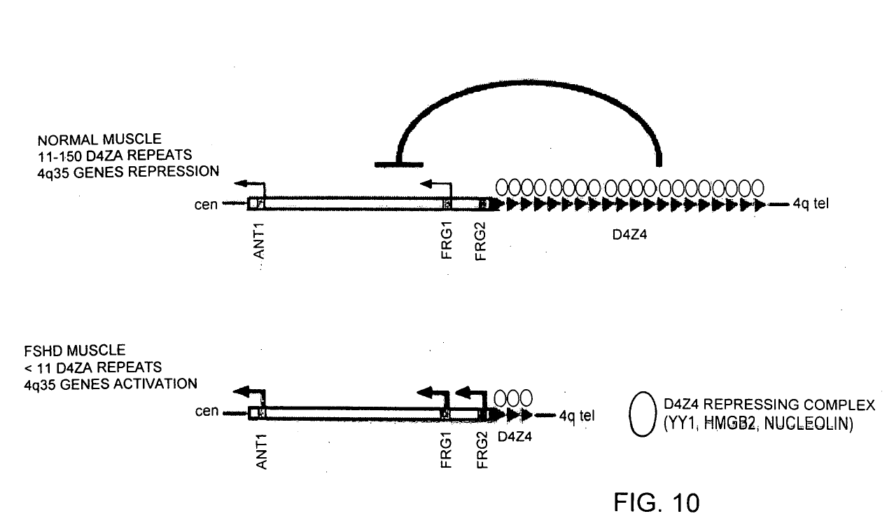

In normal individuals, the presence of a threshold number of D4Z4 repeats leads to repression of 4q35 genes by virtue of a DNA-bound multiprotein complex (MPC) that actively suppresses gene expression, referred to herein as the D4Z4 recognition complex (DRC). In FSHD patients, deletion of an integral number of D4Z4 repeats reduces the number of bound repressor complexes and consequently decreases (or abolishes) transcriptional repression of 4q35 genes. As a result, 4q35 genes are overexpressed, ultimately leading to disease onset and progression. It is shown herein that the extent of transcriptional derepression is a function of the number of deleted D4Z4 repeats (FIGS. 1E and 1F), which is also a critical determinant of disease severity (Ricci et al., 1999, Ann. Neurol. 45:751-757). The 4q35 gene overexpression model illustrated in FIG. 10 provides a possible molecular explanation for the autosomal dominant transmission of FSHD. The finding that 4q35 gene overexpression is muscle specific (FIGS. 1C and 1D) also provides an explanation for the tissue specificity of the disease. In addition to overexpression of 4q35 genes, factors such as the allelic variability of 4q35 genes, gender, and environment may also affect disease onset and severity.

It was also discovered that the DRC is composed of YY1, HMGB2, and nucleolin and associates with D4Z4 sequences both in vitro and in vivo. Significantly, decreasing the intracellular level of any one of the three DRC proteins derepressed 4q35 gene transcription, indicating that all three components are required for a functional silencing complex. In vitro binding assays demonstrated that association of the complex with D4Z4 was mediated through specific contacts between YY1 and a 27 bp element contained within D4Z4. These data suggest that YY1 is the major protein responsible for DNA binding, although other DRC components could contribute to DNA binding affinity and specificity.

The invention includes methods of identifying compounds that increase the expression or activity of DRC components, e.g., YY1, HMGB2, and nucleolin. The invention also includes methods of identifying compounds that enhance binding of YY1 and the 27 bp binding site of D4Z4 that is described herein. Compounds that demonstrate such activities (candidate compounds) are useful, e.g., to treat FSHD.

Transgenic Animals

The invention provides transgenic animals, e.g., animals that represent a model system for the study of FSHD caused by or exacerbated by the overexpression of one or more of the 4q35 genes, and for the development of therapeutic agents that decrease the expression or activity of the 4q35 genes. A 4q35 gene can be any of FRG1, FRG2, or ANT1, e.g., as described herein. A 4q35 transgene can be any transgene that increases the expression of a 4q35 gene, e.g., insertion of an additional copy of a 4q35 gene, and/or insertion of a promoter sequence that enhances expression of a 4q35 gene, e.g., an endogenous or exogenous 4q35 gene. In some embodiments, the transgene comprises an inducible promoter, allowing the expression of the 4q35 gene to be regulated at will.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats).

Any technique known in the art can be used to introduce a 4q35 transgene (e.g., FRG1, FRG2, and/or ANT1) into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Natl. Acad. Sci., USA 82:6148, 1985); gene targeting into embryonic stem cells (Thompson et al., Cell 56:313, 1989); and electroporation of embryos (Lo, Mol. Cell. Biol. 3:1803, 1983).

The present invention provides transgenic animals that carry a 4q35 transgene in all their cells, as well as animals that carry a 4q35 transgene in some, but not all of their cells. That is, the invention provides mosaic animals. The transgene can be integrated as a single transgene or in concatamers, e.g., head-to-head tandems or head-to-tail tandems. The transgene can also be selectively introduced into and activated in a particular cell type (Lasko et al., Proc. Natl. Acad. Sci. USA 89:6232, 1992; Gu et al., Science 265:103, 1984). The regulatory sequences required for such a cell-type specific activation will depend upon the particular cell type of interest, and will be known to those of skill in the art.

When it is desired that the 4q35 transgene be integrated into the chromosomal site of the endogenous gene, gene targeting is typically used. Briefly, vectors containing some nucleotide sequences homologous to an endogenous gene are designed for the purpose of integrating, via homologous recombination with chromosomal sequences, into the nucleotide sequence of the endogenous genome. Furthermore, expression of the transgene can be precisely regulated, for example, by an inducible regulatory sequence such as a regulatory sequence that is sensitive to certain physiological regulators, e.g., circulating glucose levels, or hormones (Docherty et al., 1994 FASEB J. 8:20-24), or the presence or absence of an inducing agent, e.g., selenium. Other suitable inducible promoters will be known to those of skill in the art.

Once transgenic animals have been generated, the expression of the recombinant 4q35 gene can be assayed utilizing standard techniques. Initial screening may be accomplished by, for example, Southern blot analysis or PCR techniques to determine whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR. Biological samples, e.g., tissue samples, can also be evaluated immunocytochemically using antibodies specific for the product of the 4q35 transgene.

For a review of techniques that can be used to generate and assess transgenic animals, skilled artisans can consult Gordon (Intl. Rev. Cytol. 115:171-229, 1989), and may obtain additional guidance from, for example: Hogan et al. “Manipulating the Mouse Embryo” (Cold Spring Harbor Press, Cold Spring Harbor, N.Y., 1986; Krimpenfort et al., Bio/Technology 9:86, 1991; Palmiter et al., Cell 41:343, 1985; Kraemer et al., “Genetic Manipulation of the Early Mammalian Embryo,” Cold Spring Harbor Press, Cold Spring Harbor, N.Y., 1985; Hammer et al., Nature 315:680, 1985; Purcel et al., Science, 244:1281, 1986; Wagner et al., U.S. Pat. No. 5,175,385; and Krimpenfort et al., U.S. Pat. No. 5,175,384 (the latter two publications are hereby incorporated by reference).

Assays for Expression of DRC Component Genes and 4q35 Genes

In some embodiments, expression of a DRC component gene, e.g., YY1, HMGBB, and nucleolin, is assayed. In other embodiments, expression of a 4q35 gene is assayed. Expression of such genes can be performed using methods known in the art. For example, Northern analysis or quantitative PCR methods can be used to assay expression at the level of RNA. Expression can also be assayed by measuring the level of a protein encoded by a gene. Protein levels can be measured using methods known in the art, e.g., Western analysis or other immunocytochemical methods. In some cases expression is determined by measuring the level of activity of a protein encoded by a gene.

Screening Assays

The invention provides methods (also referred to herein as “screening assays”) for identifying modulators, i.e., candidate or test compounds or agents such as proteins, peptides, peptidomimetics, peptoids, small molecules (e.g., small non-nucleic acid organic compounds, nucleic acids such as antisense RNAs, small interfering RNAs (siRNAs), oligonucleotides), small inorganic compounds, or other drugs that interact with, e.g., bind to, or have a stimulatory or inhibitory effect on a gene (or product thereof) of the present invention. Compounds thus identified can be used, e.g., to modulate the activity of target genes or gene products in a therapeutic protocol, to elaborate the biological function of the target gene product, or to identify compounds that disrupt normal target gene interactions. In general, a candidate compound is a compound that is a potential treatment for FSHD. A test compound is a compound that is to be tested in an assay such as those described herein.

In one embodiment, the invention provides assays for screening candidate or test compounds that interact with the D4Z4 binding element (DBE). In some embodiments, the invention provides assays for screening candidate or test compounds that bind to or modulate the suppressive activity of the D4Z4 binding element. In general, the compounds modulate the binding of DRC or a DRC component to the sequence that is defined as the DBE (see Example 3, infra), for example, the compound may increase binding. Increased binding can be identified as increased binding affinity or decreased rates of disassociation of the DBE and the DRC (or a DRC component).

In some embodiments, the methods described herein include administering a compound, e.g., a test compound, to a transgenic animal model of FSHD as described herein, and evaluating the animal model to see if the compound is effective in treating or preventing FSHD. For example, in some embodiments, an animal model exhibiting later onset of FSHD can be used. The onset of FSHD may be manipulated by altering the levels of overexpression of the 4q35 gene, as higher levels of overexpression may be correlated with earlier onset of symptoms; alternatively, a dose-dependent inducible promoter can be used. The test compound can be administered to the animal prior to the development of symptoms of FSHD as described herein. A test compound that is effective in treating, reducing, preventing or delaying the development of a symptom of FSHD can be considered a candidate compound. In other embodiments, an animal model already exhibiting symptoms of FSHD can be used; after administration of the test compound, the animal is evaluated for any positive effects on a symptom of the FSHD.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, nonpeptide oligomer or small molecule libraries of compounds (Lam, K. S. (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406, Cwirla et al. (1990) Proc. Natl. Acad. Sci. 87:6378-6382; Felici (1991)1 Mol. Biol. 222:301-310; Ladner supra.).

In one embodiment, an assay is a cell-based assay in which a cell that expresses a particular protein or biologically active portion thereof is contacted with a test compound, and the ability of the test compound to modulate activity of the protein is determined. The cell, for example, can be of mammalian origin, e.g., human. In some cases the cell is a muscle cell. In some embodiments, the cell is a cell such as a muscle cell from a subject that has been diagnosed with FSHD.

The ability of the test compound to modulate binding of DBE to DRC (or a DRC component) can also be evaluated. Similarly, the ability of a test compound to bind to a DBE can be evaluated. Such assays can be accomplished, for example, by coupling the compound, e.g., the substrate, with a radioisotope or enzymatic label such that binding of the compound, e.g., the substrate, can be determined by detecting the labeled compound, e.g., substrate, in a complex. Alternatively, a DBE or DRZ component could be coupled with a radioisotope or enzymatic label to monitor the ability of a test compound to modulate binding of DBE and a DRZ or DRZ component in a complex. For example, compounds can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a DBE or DRC with or without the labeling of any of the interactants can be evaluated. For example, a microphysiometer can be used to detect the interaction of a compound without the labeling of either the compound or the substrate (e.g., DBE or DRC). McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and substrate.

In yet another embodiment, a cell-free assay is provided in which a protein or biologically active portion thereof is contacted with a test compound and the ability of the test compound to bind to the protein or biologically active portion thereof is evaluated. Biologically active portions of the proteins to be used in assays of the present invention include fragments which participate in interactions with molecules, e.g., fragments with high surface probability scores.

Cell-free assays involve preparing a reaction mixture of the target DBE sequence or target DRC (or DRC component) and the test compound under conditions and for a time sufficient to allow the two components to interact and bind, thus forming a complex that can be removed and/or detected. The interaction between two molecules can also be detected, e.g., using fluorescence energy transfer (FET) (see, for example, Lakowicz et a 1., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a DBE to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either the DBE or DRC (or DRC component), an antibody raised against a DRC (or DRC component), or its target molecule to facilitate separation of complexed from uncomplexed forms of one or both of the components (DBE or DRC), as well as to accommodate automation of the assay. Binding of a test compound to a DBE or DRC, or interaction of a DBE and DRC in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or DRC (or DRC component) protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of binding or activity (e.g., of DBE and DRC) determined using standard techniques.

Other techniques for immobilizing either a DBE or DRC on matrices include using conjugation of biotin and streptavidin. Biotinylated molecules can be prepared from biotin: NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with DBE or DRC (or DRC component) molecules but which do not interfere with binding between the DBE and DRC (or DRC component). Such antibodies can be derivatized to the wells of the plate, and unbound target or DBE or DRC (or DRC component) trapped in the wells by antibody conjugation. Methods for detecting such complexes, in addition to those described above for the GST-immobilized complexes, include immunodetection of complexes using antibodies reactive with the DBE or DRC (or DRC component), as well as enzyme-linked assays which rely on detecting an enzymatic activity associated with the DBE or DRC (or DRC component) protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the DBE or DRC (or DRC component), or biologically active portion thereof with a known compound which binds DBE or DRC (or DRC component) to form an assay mixture, contacting the assay mixture with a test compound, and determining the ability of the test compound to interact with a DBE or DRC (or DRC component), wherein determining the ability of the test compound to interact with a DBE or DRC (or DRC component) includes determining the ability of the test compound to preferentially bind to DBE or DRC (or DRC component), or biologically active portion thereof, or to modulate the activity of a target molecule, as compared to the known compound.

The DBE or DRC (or DRC component) can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules.

To identify compounds that interfere with the interaction between the DBE or DRC (or DRC component) and its cellular or extracellular binding partner(s) (in addition to the DBE or a DRC component described herein), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the DBE or DRC (or DRC component) and its cellular or extracellular binding partner. Control reaction mixtures are incubated without the test compound or with a placebo. The formation of any complexes between the DBE or DRC (or DRC component) and the cellular or extracellular binding partner is then detected. The formation of a complex in the control reaction, but not in the reaction mixture containing the test compound, indicates that the compound interferes with the interaction of the DBE or DRC (or DRC component) and the interactive binding partner. Additionally, complex formation within reaction mixtures containing the test compound and normal DBE or DRC (or DRC component) can also be compared to complex formation within reaction mixtures containing the test compound and DBE or DRC (or DRC component). This comparison can be important in those cases wherein it is desirable to identify compounds that disrupt interactions of mutant but not normal target gene products.

These assays can be conducted in a heterogeneous or homogeneous format. Heterogeneous assays involve anchoring either the target DBE or DRC (or DRC component) onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the DBE or DRC (or DRC component) and the binding partners (including interactions between DBE and DRC), e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target DBE or DRC (or DRC component), is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected. Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the DBE or DRC (or DRC component) and the interactive cellular or extracellular binding partner product (for example, when the interaction between DBE and DRC is assayed) is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt the interaction can be identified.

Identification of Subjects Having or at Risk For FSHD

In some aspects, subjects are identified who are at risk for or have FSHD, e.g., by the clinical assessment of symptoms as described hereinabove, or genetically, by the presence of a diminished number of D4Z4 repeats. Methods of identifying such individuals are known in the art. These subjects may be treated for FSHD using a compound identified by the methods described herein, or a biological sample (e.g., a cell or tissue sample) may be obtained from the subject. In some cases, a cell is obtained, e.g., to evaluate a test compound or to determine whether an individual is at risk for or has FSHD. The cell can be any type of cell from the subject. In general, the cell is a muscle cell. Methods of culturing muscle cells are known in the art.

Pharmaceutical Compositions

The compounds that are identified as useful for treating FSHD by the methods disclosed herein can be incorporated into pharmaceutical compositions. Such compositions typically include compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents that modulate expression or activity. An agent may, for example, be a small molecule. For example, such small molecules include, but are not limited to, peptides, peptidomimetics (e.g., peptoids), amino acids, amino acid analogs, polynucleotides (e.g., antisense oligonucleotides or siRNAs), polynucleotide analogs, nucleotides, nucleotide analogs, organic or inorganic compounds (i.e., including hetero-organic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Methods of Treatment

The present invention provides for both prophylactic and therapeutic methods of treating a subject at risk of (or susceptible to) FSHD. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules (including small non-nucleic acid organic molecules and small inorganic molecules), peptides, antibodies, ribozymes, small interfering RNAs (siRNA), and antisense oligonucleotides.

In one aspect, the invention provides a method for preventing in a subject, FSHD by administering to the subject an agent that binds to a DBE or enhances the interaction between a DBE and DRC (or DRC component). A compound may also increase the expression of at least one 4q35 gene. Subjects at risk for a disease that is caused or contributed to by aberrant binding to a DBE (e.g., by a DRC) can be identified by, for example, any or a combination of diagnostic or prognostic assays as described herein. Administration of a prophylactic agent can occur prior to the manifestation of symptoms characteristic of FSHD, such that a disease or disorder is prevented or, alternatively, delayed in its progression. The appropriate agent can be determined based on screening assays described herein.

It is possible that the use of antisense, ribozyme, siRNA, and/or triple helix molecules that bind to, e.g., a DBE, can ameliorate or prevent symptoms of FSHD, e.g., by repressing expression of at least one 4q35 gene. The identified compounds that affect DBE or DRC (or DRC component) can be administered to a patient at therapeutically effective doses to prevent, treat or ameliorate FSHD. A therapeutically effective dose refers to that amount of the compound sufficient to result in amelioration of symptoms of the disorders. Toxicity and therapeutic efficacy of such compounds can be determined by standard pharmaceutical procedures as described above. The data obtained from the cell culture assays and animal studies can be used in formulating a range of dosage for use in humans. The dosage of such compounds preferably lies within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate, e.g., binding between DBE or DRC (or DRC component) is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, 1994, Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound can be readily monitored and used in calculations of IC50.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC50. An rudimentary example of such a “biosensor” is discussed in Kriz, D. et al (1995) Analytical Chemistry 67:2142-2144.

In one embodiment, the agent binds to a DBE or increases the interaction between a DBE and DRC (or DRC component). Such a compound may decrease the activities of one or more 4q35 gene products. Examples of such inhibitory agents include gene products that have decreased activity compared to a wild type gene product or a nucleic acid encoding a gene product with such reduced activity. In another embodiment, the agent inhibits one or more activities of a 4q35 gene product, e.g., by increasing the interaction between DBE or DRC (or DRC component), or by binding to a DBE. Examples of such inhibitory agents include antisense nucleic acid molecules, antibodies, and compounds that increase expression of a DRC component. These modulatory methods can be performed in vitro (e.g., by culturing the cell with the agent) or, alternatively, in vivo (e.g., by administering the agent to a subject). As such, the present invention provides methods of treating an individual afflicted with FSHD. In one embodiment, the method involves administering an agent (e.g., an agent identified by a screening assay described herein), or combination of agents that modulates (e.g., up regulates or down regulates) a expression or activity of a DRC component or a 4q35 gene product.

A decrease in activity of a 4q35 gene is desirable in FSHD subjects in which a 4q35 is abnormally upregulated.

This invention is further illustrated by the following examples which should not be construed as limiting. The contents of all references, patents and published patent applications cited throughout this application are hereby incorporated by reference.

### EXAMPLES

### Example 1

### Experimental Procedures

Cell Lines, Cell Culture

HeLa and C2C12 mouse myoblast cell lines were obtained from the American Type Culture Collection (ATCC). GM10115 (Coriell Cell Repositories) is a human/chinese hamster somatic cell hybrid retaining human chromosome 4. HeLa and C2C12 cell lines were routinely cultured in a humidified atmosphere at 37° C. with 5% CO₂ in Dulbecco's modified Eagle's medium (DMEM) supplemented with 10% fetal calf serum (FCS), 2 mM glutamine, and 70 μg/ml gentamycin. GM10115 cells were cultured in the same medium containing 0.2 mM proline.

Antibodies

Primary antibodies used for immunoblotting and chromatin immunoprecipitation experiments were obtained as follows: α-HMGB2 from BD Transduction Laboratories, and α-tubulin from Sigma. The 7G2 monoclonal α-nucleolin antibody was kindly provided by S. Pinol-Rama.

Purification of the D4Z4 Binding Protein

HeLa nuclear extracts were prepared according to standard protocols (Ausubel et al., 2001, Current Protocols in Molecular Biology, New York, John Wiley & Sons) and dialyzed against dialysis buffer (20 mM HEPES (pH 8), 20% glycerol, 100 mM EDTA, 0.2 mM phenylmethylsulphonyl fluoride (PMSF), 0.5 mM dithiothreitol (DTT)). Sixty milliliters of nuclear extract (360 mg of protein) was fractionated on 30 ml of P11 phosphocellulose (Whatman) using a 0.1-1 M KCl step gradient in dialysis buffer. The active fraction was dialyzed against dialysis buffer and fractionated on 1 ml of DEAE-Sepharose (Pharmacia) using 0.1-1 M KCl linear gradient in dialysis buffer. Active fractions were pooled, dialyzed against binding buffer 50 (20 mM HEPES (pH 8), 10% glycerol, 50 mM KCl, 0.2 mM EDTA, 0.2 mM PMSF, 0.5 mM DTT), and loaded onto a DNA affinity resin prepared by coupling the D4Z4 minimal binding site to CNBr-activated Sepharose 4B (Pharmacia) according to standard protocols (Ausubel et al., 2001). This column was eluted using a 0.05-1 M KCl linear gradient in binding buffer 50. The active fractions were pooled, dialyzed against binding buffer 150 (20 mM HEPES (pH8), 10% glycerol, 150 mM KCl, 0.2 mM EDTA, 0.2 mM PMSF, 0.5 mM DTT), and fractionated on a new D4Z4 affinity column using a 0.15-1 M KCl linear gradient in binding buffer 150. For peptide sequencing, 0.1 ml of a final active fraction (representing approximately 1/20 of the final yield) was resolved by 12% SDS-PAGE. Following staining with Silver Staining Plus (BioRad), the protein bands were excised from the gel and analyzed by MALDI-TOF Mass Spectrometry at the University of Massachusetts Medical School Protein Microsequencing and Mass Spectrometry Center.

Immunoblot Analysis

Total lysates were obtained by boiling the samples in 1×SDS sample buffer (Laemmli buffer), fractionated onto SDS-PAGE, and electroblotted to PVDF filters (Millipore) in 25 mM Tris base, 192 mM glycine, 20% v/v methanol (pH 8.3) using a TransBlot cell (BioRad). The membrane was blocked in 5% nonfat dry milk (Carnation) in TBST (20 mM Tris-HCl (pH 7.5), 150 mM NaCl, 0.1% Tween-20) for 60 minutes, followed by incubation with the primary antibody for 60 minutes. After three serial washings with TBST for 10 minutes, the antigen antibody complexes were visualized with the appropriate secondary antibody (Jackson ImmunoResearch) conjugated to horseradish peroxydase and a chemiluminescent system as recommended by the manufacturer (NEN).

Electro-Mobility Shift Assay (EMSA) and Supershift

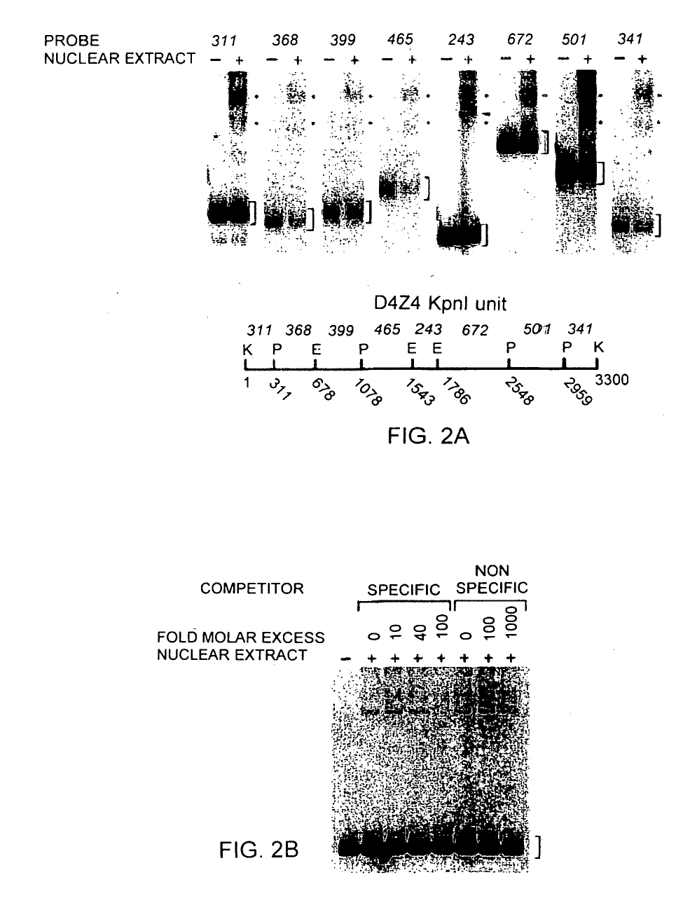

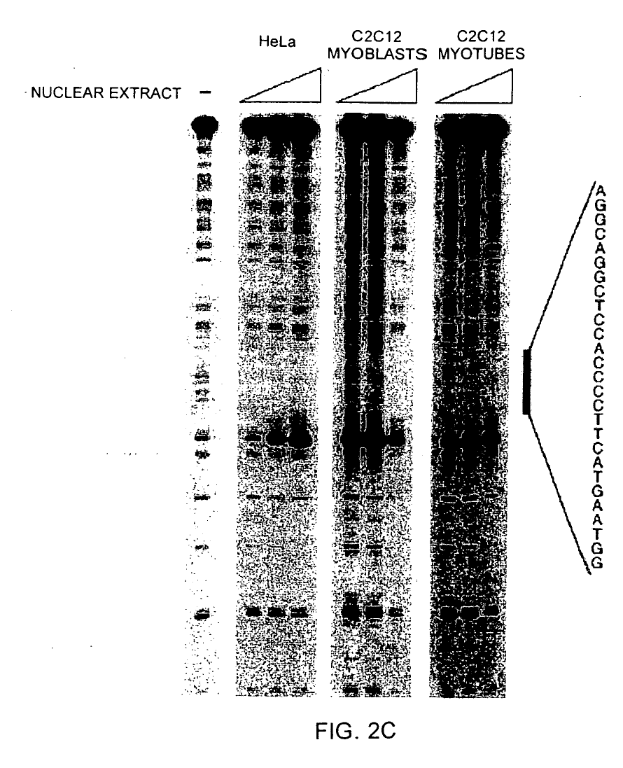

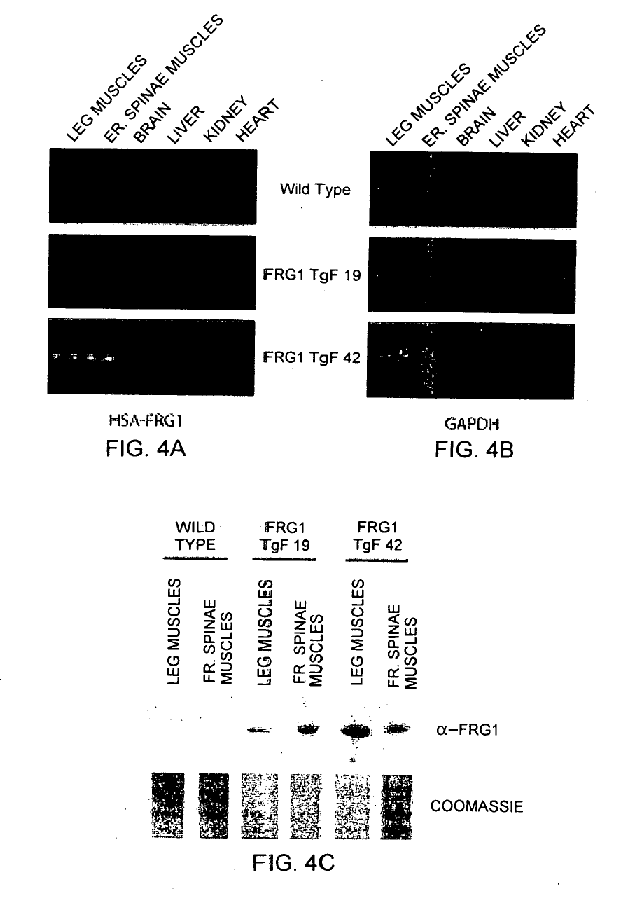

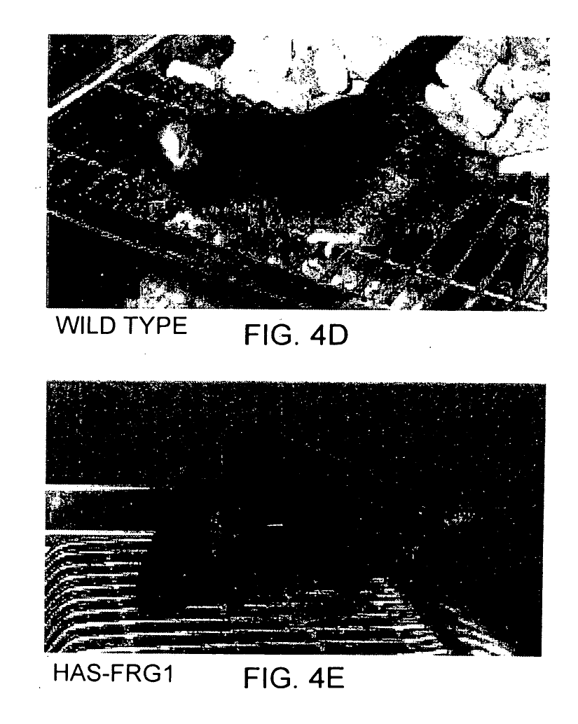

The Kpnl-D4Z4 unit plasmid, used to generate EMSA probes for the experiments shown in FIGS. 2A-2C, was kindly provided by L. Felicetti. For the EMSA experiments shown in FIG. 4, oligonucleotide probes were prepared by end-labeling double-stranded oligonucleotides covering the region of the minimal binding site. The sequences of the oligonucleotides were as follows: WT, 5′-CTCACCGCCATTCATGAAGGGGTGGAGCCTGCCTG-3′ (SEQ ID NO:1); MUT, 5′-CTCACCGCCgccCATGAAGGGGTGGAGCCTGCCTG-3′ (SEQ ID NO:2) (mutated nucleotides are shown in italics). DNA binding reactions were carried out in 20 μl of 10 mM HEPES (pH 8), 10% glycerol, 50 mM KCl, 0.1 mM EDTA, 0.25 mM DTT, 0.1 mg/ml poly (dl-dC), 0.1 mg/ml bovine serum albumin, 20,000 cpm of radiolabeled probe. The samples were incubated for 30 minutes on ice in the presence of the indicated nuclear extract or protein fraction. For competition assays, unlabeled probes were added to the reaction mixture at the same time as the labeled probe. For supershift assays, a binding reaction was assembled in the absence of radiolabeled probe. Antibodies were added to the binding reaction and the samples were incubated 15 minutes at room temperature. Radiolabled probe was added and the samples were incubated for additional 30 minutes on ice. Reaction mixtures were loaded in the cold room onto 6% native polyacrylamide gels containing 0.5×Tris-borate-EDTA (TBE) that had been preelectrophoresed for 30 minutes at 20 mA. After electrophoresis for 1.5-2 hours at 20 mA, the gels were dried and exposed for autoradiography.

DNase I In Vitro Footprinting

DNase I in vitro footprinting experiments were performed essentially as described in Ausubel et al. (2001, supra). D4Z4-243 was cloned into pBluescript SK⁺ vector (Stratagene) for use as a probe. Briefly, the vector was digested with Asp718, labeled with Klenow in the presence of [α-³²P]dATP and [α-³²P]dCTP, digested with SacI, and gel purified DNA binding reactions were carried out as for EMSA in the presence of different amounts of nuclear extracts. After a 30 minutes incubation on ice, 0.3 units of DNase I (RQ1, Promega) was added and the samples incubated 1 minute at room temperature. Reactions were stopped, and phenol was extracted, precipitated, and resuspended in formamide loading buffer. Samples were loaded onto 6% denaturing polyacrylamide gels along with sequencing reactions that were prepared as described in Ausubel et al. (2001, supra). After electrophoresis the gels were dried and exposed for autoradiography.

Chromatin Immunoprecipitation (ChIP)

ChIP analysis was performed according to published procedures (Boyd et al., 1998, Proc. Nat. Acad. Sci. USA 95:13887-13892; the protocol was kindly provided by P. J. Farnham). Quantitative PCR was performed as previously described (Orlando and Paro, 1993, cell 75:1187-1198) using an aliquot (1/30) of the purified DNA. PCR reactions contained [α³²P]dATP (2.5 μCi for each 25 μl reaction), The PCR products were detected by autoradiography after separation on a 6% polyacrylamide gel. Sequences of the oligonucleotides used are as follows: D4Z4 region, DBS-I (5′-AGGCCTCGACGCCCTGGGTC-3′; SEQ ID NO:3) and DBS-II (5′-TCAGCCGGACTGTGCACTGCGGC-3′; SEQ ID NO:4); p13E-11 region, p13-I (5′-AGCCCTGCCACAGGCTTCTGTG-3′; SEQ ID NO:5) and p13-11(5′-AGTGCTTATGCCTGAGGAATCTG-3′; SEQ ID NO:6); FRG1, FRG1-1f (5′-TCTACAGAGACGTAGGCTGTCA-3′; SEQ ID NO:7) and FRG1-1rb 9 (5′-CTTGAGCACGAGCTTGGTAG-3′; SEQ ID NO:8).

RNA Extraction and RT-PCR

Total RNA was prepared using the TRI Reagent (Sigma) according to the manufacturer's instructions for the isolation of RNA for RT-PCR. Purified RNA was treated with RNase-free DNase I (Promega) to remove residual DNA, and 1 μg of purified DNA-free RNA was used for first-strand sDNA synthesis with SuperScript II Rnase H⁻RT (RT; Invitrogen) according to the manufacturer's instructions. Quantitative PCR was performed as previously described (Orlando and Paro, 1993, supra) using an aliquot (1/20) of the RT reaction. PCR reactions contained [α-³²P]dATP (2.5 μl for each 25 μl reaction), and the PCR products were detected by autoradiography after separation on a 6% polyacrylamide gel. Sequences of the oligonucleotides used are as follows: FRG1, FRG-1f (5′-TCTACAGAGACGTAGGCTGTCA-3′; SEQ ID NO:9) and FRG1-1rb (5′-CTTGAGCACGAGCTTGGTAG-3′; SEQ ID NO:10); FRG2, EX2BF (5′-CCAGAGTCCAGCTCATATCG-3′; SEQ ID NO:11) and SSC8 (5′-CTCACAGGTAAGTGGAGAATGG-3′; SEQ ID NO:12); ANT1, ANT1 (5′-GTGCATTAAGTGGTCTTTATT-3′; SEQ ID NO:13) and ANT2 (5′-TGTGGTTTAATAGACTATTCCTA-3′; SEQ ID NO:14); GAPDH, G3PDH 5′ (5′-ACCACAGTCCATGCCATCAC-3′; SEQ ID NO:15); and G3PDH 3′ (5′-TCCACCACCCTGGTTGCTGTA-3′; SEQ ID NO:16). FRG1 and ANT1 primers are specific for 4q35. Sequences that are closely related to FRG2 are present on chromosomes 1, 4, 8, 10, and 20; however, the FRG2 sequence at 4q35 is the only one resistant to digestion with Tail and Hhal. Therefore, to obtain a 4q35-specific signal, FRG2 PCR products were digested with both enzymes before electrophoresis.

Purification of GST-YY1

GST-YY1 expression vectors were kindly provide by Y. Shi, E. Seto, A. Usheva, D. M. Margolis, and T. Shenk. For purification, saturated cultures of E. coli expressing the GST-YY1 fusion protein were diluted 1:20 with fresh medium and incubated at 37° C. until the OD at 600 nm was 0.6. Isopropyl-beta-D-thiogalactopyranoside (IPTG) was added to a final concentration of 0.2 mM, and the bacteria were incubated at 37° C. for 4 hours. Bacteria were harvested, washed once with STE (10 MM Tris (pH 8.0), 150 mM NaCl, 1 mM EDTA), and resuspended in STE containing 100 μg/ml of lysozyme. Following a 15 minute incubation on ice, DTT was added to a final concentration of 5 mM. N-Lauryl Sarcosine was added to a final concentration of 1.5% (from 10% stock in STE), and the bacteria were sonicated on ice until the solution was clear (˜1 min). Triton X-100 was added to a final concentration 3%. Following centriguation, the supermutant was added to glutathione agarose beads (50% suspension in STE) for affinity purification. Fusion proteins were eluted using 75 mM HEPES (pH 7.4), 150 mM NaCl, 10 mM reduced glutathione, 5 mM DTT, and 2% N-octyl glucoside. Purified GST-YY1 was dialyzed again dialysis buffer (20 mM HEPES [pH 8], 20% glycerol, 100 mM KCl, 0.2 mM EDTA, 0.2 mM PMSF, 0.5 mM DTT).

Antisense Morpholino Oligonucleotides

Morpholino oligonucleotides (GeneTools) spanning the sequence of the AUG translational start site of human YY1 (5′-CATGGCTGAGGGCTCCGCCGCCACG-3′; SEQ ID NO:17); HMGB2 (5′-GGGTCTCCTTTACCCATGTTGACAG-3′; SEQ ID NO:18); nucleolin (5′-GCGAGCTTCACCATGATGGCGGCGG-3′; SEQ ID NO:19), or standard control oligonucleotides (5′-CCTCTTACCTCAGTTACAATTTATA-3′; SEQ ID NO:20) were introduced into HeLa cells according to the manufacturer's special delivery protocol (Morcos, 2001). Oligonucleotide delivery was repeated at 48 hours intervals for a total of five times. Cells were maintained at exponential growth throughout the entire procedure. Immunoblotting and quantitative RT-PCR analysis was performed 48 hours after the last delivery.

Repression Assay

The vector pcDNA 3.1 (Invitrogen) was digested with Kpnl and Smal, blunt-ended, and relegated to obtain CMV-Neo. Tandem repeats of the DBE sequence were produced as described (Ausubel et al., 2001) and cloned into the Nhel site of CMV-Neo to obtain CMV-DBE-Neo. A spacer sequence equivalent in length to six DBE repeats was generated by digesting pBluscript SK+ (Stratagene) with Spel and Pvull and was cloned into Nhel/HindIII-digested CMV-Neo that had been blunt-ended at the HindIII site to obtain CMV-Spacer-Neo. Each construct was verified by sequencing.

For the repression assay, each construct was linearized with Pvul and transfected into HeLa cells in 6-well plate with Effectene according to the manufacturer's instructions (Qiagen). Forty-eight hours after transfection, one-third of each well passed in 100 mm plates, and G418 (1 mg/ml, Calbiochem) was added to the media. Two weeks later, colonies were fixed and stained with Giemsa, and the number of G418-resistant cells counted. Experiments were repeated in triplicate, using two different plasmid preparations.

### Example 2

### Inappropriate Overexpression of 4q35 Genes in FSHD Dystrophic Muscle

To investigate whether altered expression of 4q35 genes is the underlying basis of FSHD, human muscle samples were collected from normal individuals and patients affected by FSHD as well as several other muscular dystrophies type 2 (LGMD). Expression of three 4q35 genes, FSHD region gene 1 (FRG1), FSHD region gene 2 (FRG2), and the adenine nucleotide translocator-1 gene (ANT1), was analyzed by RT-PCR.

FIG. 1A shows that expression of FRG2, the gene most proximal to D4Z4, was undetectable in normal muscle but was present at a significant level in all three FSHD muscle samples. Expression of two other 4q35 genes, FRG1 and ANT1, was detected in all muscle samples, but overexpressed only in FSHD muscle. Quantitation by phosphoimager analysis revealed, first, that in normal muscle the expression level of 4q35 genes increased with distance from D4Z4 and, second, that in FSHD muscle the level of overexpression varied inversely with distance from D4Z4. Glyceraldehyde Phosphate Dehydrogenase (GAPDH), which is not at 4q35, was expressed equivalently in all muscle samples. Significantly, FIG. 1C shows that in lymphocytes from FSHD patients, expression of FRG2, FRG1, and ANT1 was equivalent to that observed in normal tissue, indicating that overexpression of 4q35 genes in FSHD is muscle specific. Expression of 4q35 genes was also normal in DMD, ALS, and LGMD muscle samples (FIG. 1D), indicating that misregulated 4q35 gene expression is not a general characteristic of muscular dystrophies.

The number of D4Z4 repeats is critical determination of disease severity (Ricci et al., 1999). The results of FIGS. 1A and 1C-1D raised the possibility that inappropriate overexpression of 4q35 genes located upstream of D4Z4 is the underlying basis of FSHD. A prediction of this model is that 4q35 gene overexpression would be increased when the number of D4Z4 repeats is decreased. To test this prediction, expression of FRG2 in muscle biopsies from six additional FSHD patients carrying varying numbers of D4Z4 repeats was analyzed. The results of FIG. 1E-1F clearly demonstrate that the level of FRG2 overexpression is indeed inversely related to the number D4Z4 repeats. On the basis of FIGS. 1A-1F, it can be concluded that 4q35 gene mRNAs and presumably proteins are overexpressed in FSHD muscle in a manner inversely related to distance from D4Z4 and the number of D4Z4 repeats.

### Example 3

### Detection of Nuclear Activity that Binds to D4Z4

Mechanism by which D4Z4 could suppress 4q35 gene transcription is through interaction with a cellular factor (or factors) that silences the expression of nearby genes. This possibility was tested by analyzing the interaction between D4Z4 and nuclear proteins using an electrophoretic mobility shift assay (EMSA). Probes were generated by ³²P-end-labeling eight restriction enzyme-digested fragments that spanned the entire D4Z4 sequence. FIG. 2A shows that one DNA fragment, termed D4Z4-243, supported formation of a specific complex. This complex was abolished by addition of increasing amounts of D4Z4-243 DNA but not by non-specific DNA (FIG. 2B). A comparable D4Z4-243 binding activity was also detected in nuclear extracts prepared from several human and mouse myogenic cell lines (see below and data not shown).

Deoxyribonuclese (DNase 1) footprinting was used to map the binding site within D4Z4-243. FIG. 2C shows that following incubation with nuclear extracts from HeLa cells, C2C12 myoblasts, or C2C12 myotubes, a 27 basepair (bp) sequence (CCATTCATGAAGGGGTGGAGCCTGCCT; SEQ ID NO:21) within D4Z4-243 was protected from DNase I digestion. This 27 bp sequence, is defined as the D4Z4 binding element (DBE).

### Example 4

### The DBE is a Transcriptional Repression Element

One explanation for the results presented above is that the DBE is a transcriptional repression element that, when deleted in FSHD, leads to overexpression of 4q35 genes. To test this hypothesis, the ability of the DBE to suppress transcription of reporter gene expressed from the strong cytomegalovirus (CMV) promoter was analyzed. Analogous assays have been used previously to identify positive and negative transcriptional regulatory elements (see, for example, Chung et al., 1997).

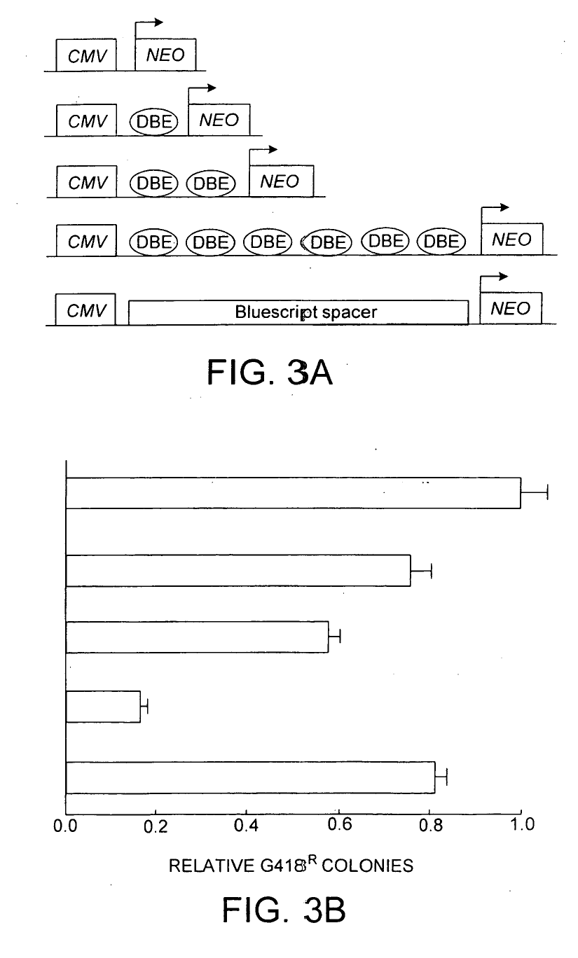

To perform the assays, a series of plasmids were constructed in which increasing numbers of DBEs were inserted between the CMV promoter and the neomycin-resistance gene. The plasmids were stably transfected into HeLa cells, and the number of G418-resistant colonies was quantitated. As shown in FIGS. 3A-3B, the number of inserted DBEs inversely correlated with the number G418-resistant colonies: in the presence of one, two, or six DBEs, the number of G418-resistant colonies decreased by 25%, 45%, and 86%, respectively. Significantly, insertion of a nonrelated spacer sequence had no effect, indicating that the decrease in colony number was not simply due to increased distance between the CMV promoter and the reporter gene. These data indicate that the DBE within D4Z4 is a transcriptional repression element.

### Example 5

### Identification of a Multiprotein Complex that Binds to the DBE

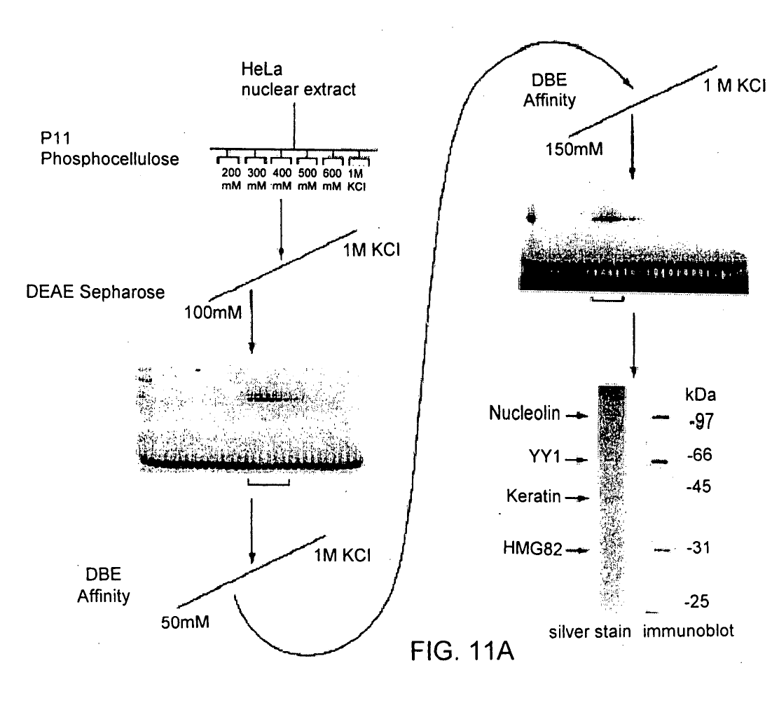

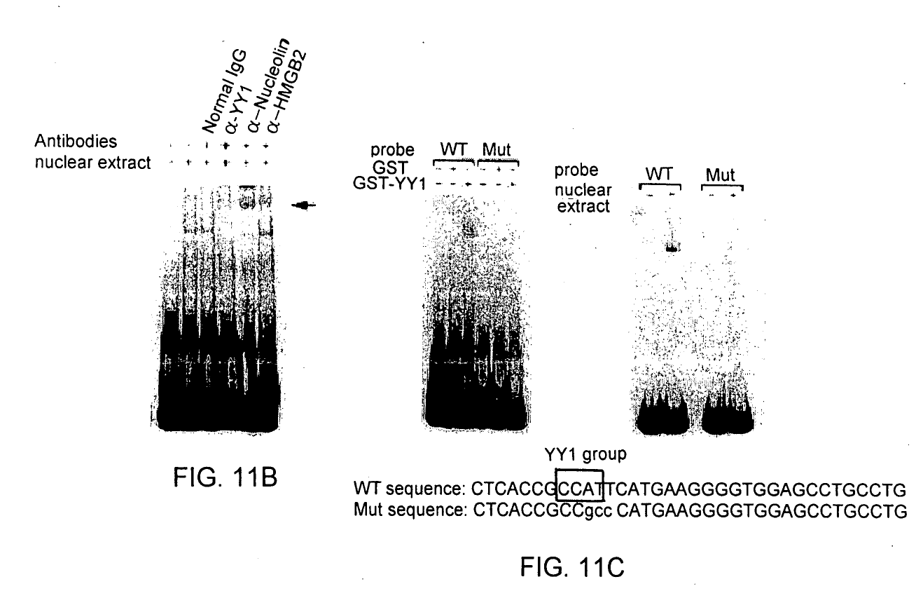

To identify and characterize proteins that bind to the DBE, the D4Z4 recognition complex (DRC) was biochemically purified using sequential chromatography of HeLa nuclear extracts on P11 Phosphocellulose, DEAE-Sepharose, and DBE-Agarose (see FIG. 11A, schematic). The purified DRC was fractionated on an SDS-Page gel, and the polypeptides were detected by silver staining, excised, and microsequenced by mass spectrometry. FIG. 11A shows that four bands were present in the active fraction: YY1, a multifunctional repressor/activator (reviewed in Thomas and Seto, 1999); HMGB2, a member of a family of nonhistone chromatin-associated proteins (reviewed in Thomas and Travers, 2001); nucleolin; and keratin. Keratin is a common contaminant of mass spectrometric analysis (Eng et al., 1994) and was not further analyzed. Immunoblot analysis confirmed the identity of YY1, HMGB2, and nucleolin (FIG. 11A).

To verify that this multiprotein complex was in fact the nuclear DBE binding activity, mobility-shift experiments were performed using antibodies directed against YY1, HMGB2, and nucleolin. FIG. 11B shows that antibodies to nucleolin and HMGB2 resulted in formation of a lower mobility complex (“supershift”), whereas addition of a control antibody had no effect. An antibody to YY1 interfered with formation of the DBE mobility shift, suggesting that association of YY1 with either a component of the DRC or the DBE itself had been disrupted.

### Example 6

### Recognition of the DBE by YY1

It was observed that the DBE contains a putative YY1 recognition sequence (CCATN; SEQ ID NO:22; Yant et al., 1995), suggesting that YY1 directly interacts with the DBE. To test this possibility, mobility-shift experiments were carried out using recombinant YY1. FIG 11C shows that recombinant GST-YY1, but not GST alone, bound the DBE probe. A DBE probe containing a mutation within the YY1 core recognition sequence was not bound by GST-YY1. Moreover, this mutant probe failed to support a mobility shift with HeLa nuclear extract (FIG. 11C, right) suggesting that elimination of YY1 binding also abolished association of the entire DRC to the DBE. These data show that YY1 has intrinsic DBE binding activity and that YY1 is the most likely DRC component to directly contact the DBE.

### Example 7

### The DRC Binds D4Z4 In Vivo

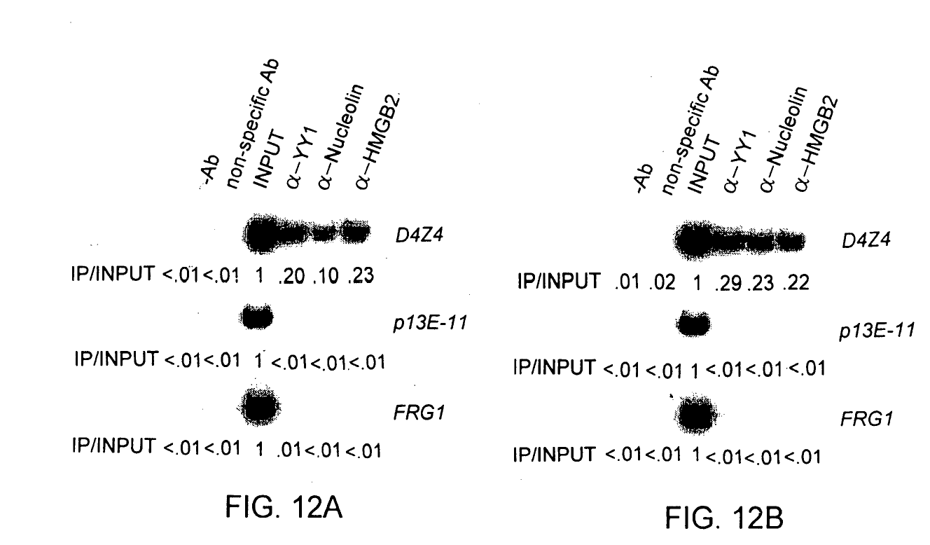

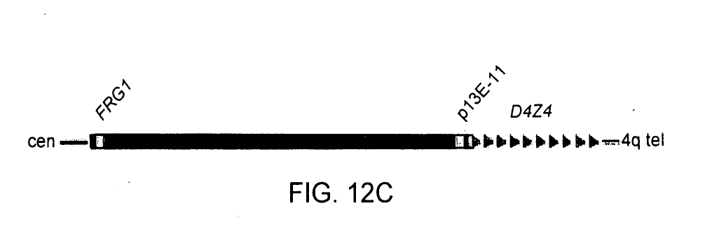

To determine whether the DRC also binds D4Z4 sequences in vivo, we performed chromatin immunoprecipitation (ChIP) experiments. HeLa cells were first treated with formaldehyde to induce crosslinks between the DBE and bound proteins. DNA-complexes were then immunopurified using antibodies directed against various DRC components, and the presence of specific DNA fragments in the immunoprecipitate was quantified by PCR. Three sets of primer pairs were used that are specific to either D4Z4, p13E-11, or FRG1. FIG. 12A shows that all three DRC components, YY1, HMGB2, and nucleolin, were associated with D4Z4 sequences. In contract, there was no detectable binding of any DRC component to the control sequences p13E-11 or FRG1.

The human genome contains sequences homologous to D4Z4 on several chromosomes in addition to 4 (Wijmenga et al., 1992; Winokur et al., 1994; Lyle et al., 1995). It was therefore possible that the interaction between the DRC and DBE detected in the ChIP assay shown in FIG. 12A did not occur at 4q35, but rather at one or more of these other chromosomal loci. To address this possibility, the ChIP assay was used to analyze binding to the DBE in a human/rodent monomchromosomal cell hybrid containing a single human chromosome 4. The rodent genome lacks D4Z4 repetitive sequences (Clark et al., 1996), and therefore in this experimental system the only genomic copy of D4Z4 is present at 4q of the human chromosome. FIG. 12B shows that in this cell line, all three DRC components were specifically associated with the D4Z4 sequences. These results indicate that in vivo the DRC in bound to D4Z4 within 4q35.

### Example 8

### Reducing the Levels of DRC Components Results in Overexpression of 4q35 Genes

The results presented above suggest that deletion of D4Z4 repeats reduces the number of DBEs and thus the number of DNA bound transcriptional repressing complexes is predicted to result in the inappropriate upregulation of 4q35 genes. To verify that the DRC is responsible for the lack of 4q35 gene expression in normal cells, antisense experiments were performed to decrease the intracellular levels of DRC components.

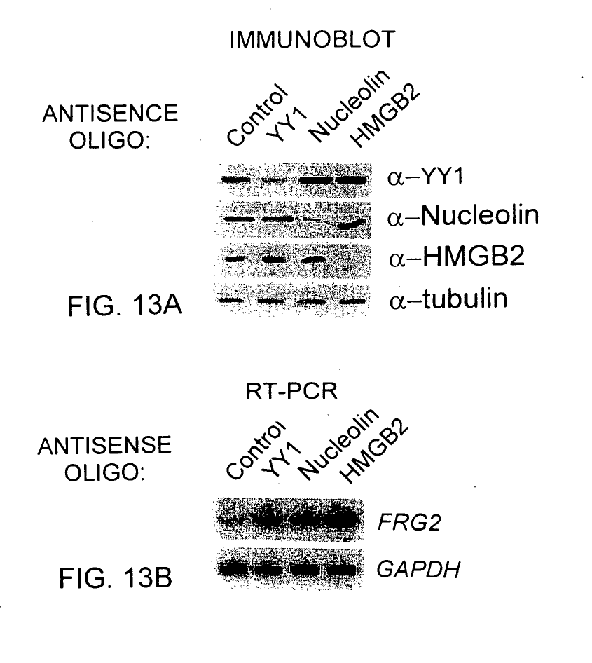

HeLa cells were transfected with morpholino oligonucleotides targeted to human YY1, HMGB2, or nucleolin mRNA. The immunoblot in FIG. 13A shows that each antisense oligonucleotide specifically reduced the level of its cognate protein. FIG. 13B shows that reducing the levels of YY1, HMGB2, or nucleolin resulted in overexpression of the 4q35 gene FRG2. Thus, reducing the levels of DRC components recapitulates the molecular event observed at 4q35 in FSHD muscle.

### Example 9

### Generation of a Mouse Showing Myopathic Features.

Transgenic mice were generated that recapitulate FSHD pathophysiology.

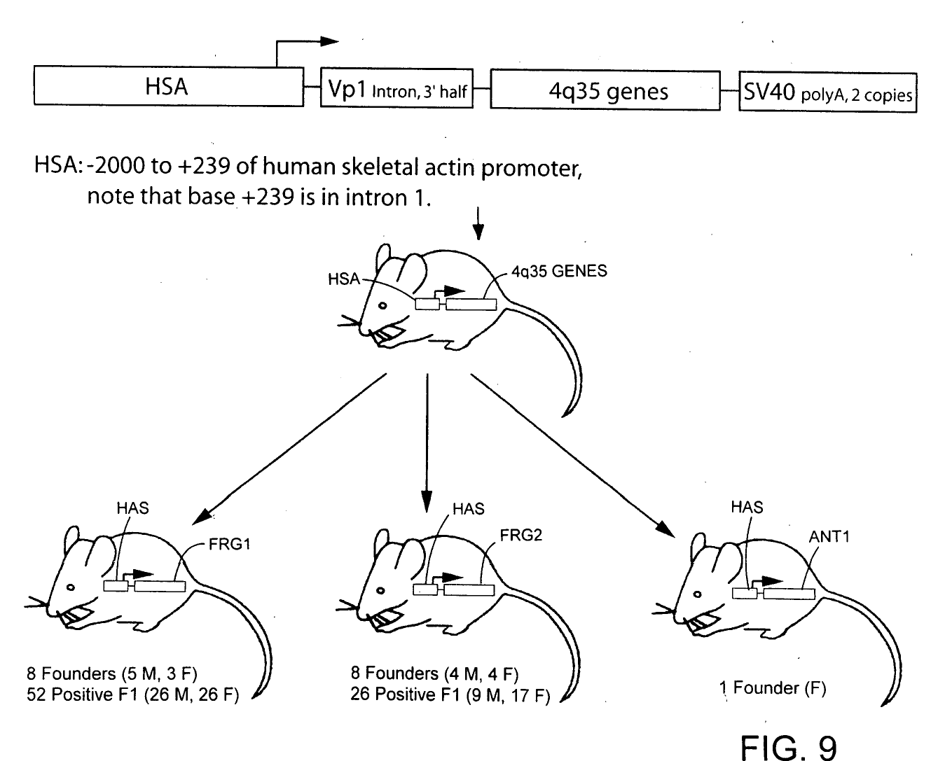

Transgenic mice over-expressing ANT1, FRG1, and FRG2, genes over-expressed in muscle tissues affected by FSHD (Gabellini et al, 2002), were generated. For this purpose, ANT1, FRG1, and FRG2 were cloned into an expression vector carrying the human skeletal actin (HSA) promoter that is expressed in all skeletal muscle fibers of the adult mouse, with minor fiber to fiber variation (Brennan, 1993). FIG. 9 shows a schematic illustration of the construct used to make the transgenic mice.

Transgenic mice were generated following standard procedures. Eight founders expressing the HSA-FRG1 transgene, seven founders expressing the HSA-FRG2 transgene, and one founder expressing the HSA-ANT1 transgene were obtained. The mouse colonies over-expressing the HSA-FRG1 and HSA-FRG2 transgenes were expanded, and the mouse colony of the HSA-ANT1 transgene is being expanded.

As shown in FIGS. 4D-E, transgenic mice over-expressing the FRG1 transgene (also referred to herein as HSA-FRG1 mice) display an abnormal phenotype. The body weight of the HAS-FRG1 mice is diminished compared to normal control mice, and severe kyphosis of the dorsal spine was clearly visible. The spine deformities associated with FSHD are thought to be due not to, or not only to, skeletal abnormalities, but to muscle weakness. Notably, FSHD patients show a lordotic posture due to an altered curvature of the vertebral column, because of the weakness of abdominal muscles.

Necropsy of the FRG1 transgenic animals revealed the absence of skeletal deformities, whereas the paravertebral muscles appeared smaller when compared to normal controls. Indeed, post-mortem, the spine showed normal curvature. As one hypothesis, the kyphotic posture of the transgenic mice is due to altered muscle function. Notably, the kyphotic phenotype is present in all transgenic mice over-expressing FRG1, and the severity correlates with the transgene expression level.

FRG1 expression profile was analyzed at either the mRNA level or the protein level in skeletal muscle, heart, brain, kidney and liver. As shown in FIGS. 4A-C, the FRG1 transgene is expressed uniquely in skeletal muscle tissues. FRG1 expression is also present in paravertebral muscles, whose weakness might be responsible for the kyphosis observed in vivo.

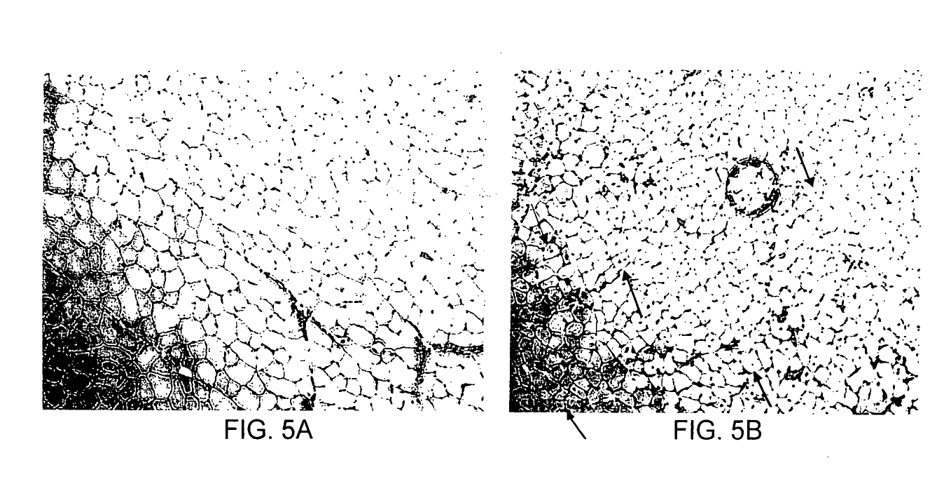

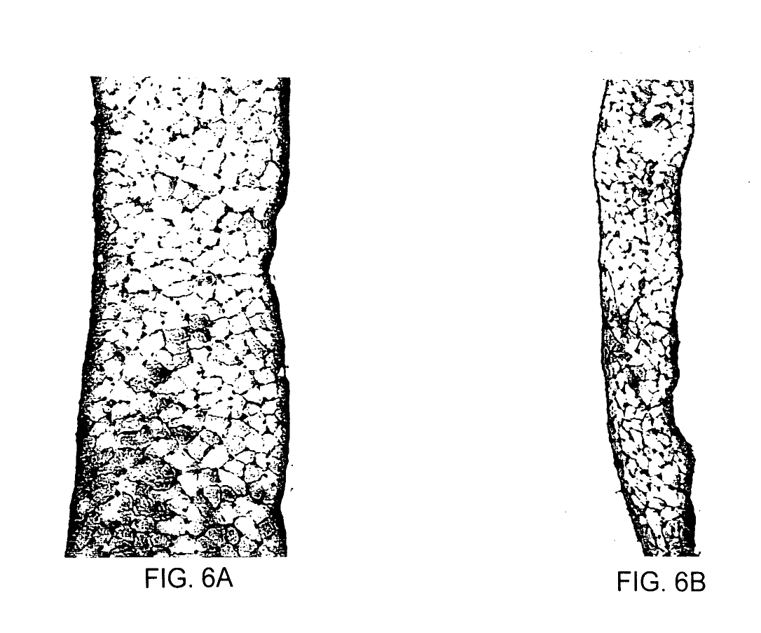

Histological analysis comparing several muscles dissected from HSA-FRG1 mice to muscles from normal control mice was also performed. As shown in FIG. 5B, sections from tibialis anterior reveals muscle fibers of various caliber and many muscle-fibers with central nuclei (arrows) that are typical of a dystrophic process; the normal control is shown in 5A. The weight of the dissected muscle was diminished when compared to normal control muscle. FIGS. 6A (normal control) and 6B (transgenic) shows the evident changes also present in the diaphragm.

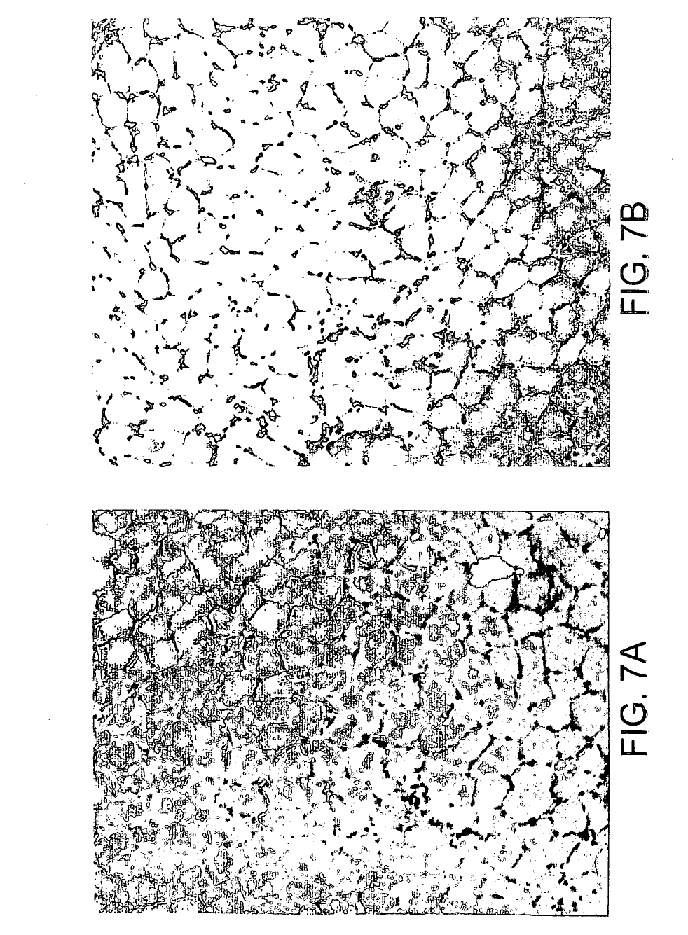

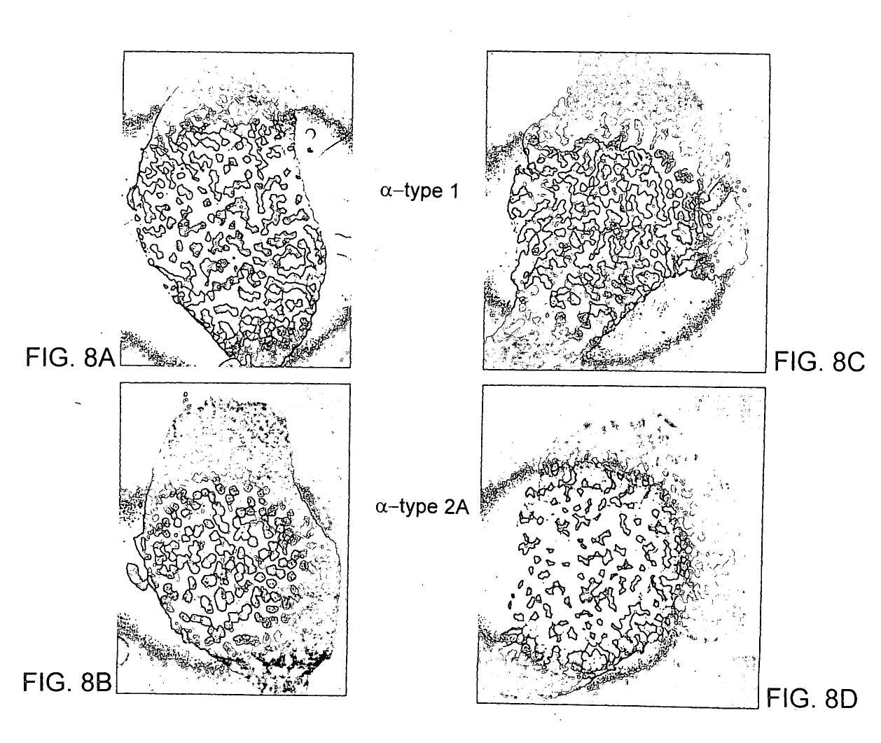

Analysis of soleus muscle revealed a different pattern: the architecture of the transgenic muscle is almost intact with very few cell with central nuclei (FIG. 7B), the muscle weight is comparable to the normal control (FIG. 7A). Interestingly, fiber-type composition clearly differs between muscles obtained from the normal (FIGS. 8A-B) and HAS-FRG1 transgenic animals (FIGS. 8C-8D). Soleus from HSA-FRG1 mouse is composed mainly of type-1 fibers (FIG. 8C), as compared to normal mice, in which the majority of the soleus is composed of type-2 fibers (FIG. 8B).

These results provide evidence of a pivotal role of FRG1 over-expression in generating a myopathic phenotype.

### REFERENCES

Ausubel, F. M., Brent, R., Kingston, R. E., Moore, D. D., Seidman, J. G., Smith, J. A., and Struhl, K. (2001). Current Protocols in Molecular Biology (New York: John Wiley & Sons).
Bauer, M. K., Schubert, A., Rocks, O., and Grimm, S. (1999). Adenine nucleotide translocase-1, a component of the permeability transition pore, can dominantly induce apoptosis. J. Cell Biol. 27, 1493-1502.
Bennett, S. T., Lucassen, A. M., Gough, S. C., Powell, E. E., Undlien, D. E., Pritchard, L. E., Merriman, M. E., Kawaguchi, Y., Dronsfield, M. J., Pociot, F., et al. (1995). Susceptibility to human type 1 diabetes at IDDM2 is determined by tandem repeat variation at the insulin gene minisatellite locus. Nat. Genet. 9, 284-292.
Blair, I. P., Adams, L. J., Badenhop, R. F., Moses, M. J., Scimone, A., Morris, J. A., Ma, L., Austin, C. P., Donald, J. A., Mitchell, P. B., and Schofield, P. R. (2002). A transcript map encompassing a susceptibility locus for bipolar affective disorder on chromosome 4q35. Mol. Psychiatry, in press.
Boyd, K. E., Wells, J., Gutman, J., Bartley, S. M., and Farnham, P. J. (1998). c-Myc target gene specificity is determined by a post-DNA binding mechanism. Proc. Natl. Acad. Sci. USA 95, 13887-13892.
Chung, J. H., Bell, A. C., and Felsenfeld, G. (1997). Characterization of the chicken β-globin insulator. Proc. Natl. Acad. Sci. USA 94, 575-580.
Clark, L. N., Koehler, U., Ward, D. C., Wienberg, J., and Hewitt, J. E. (1996). Analysis of the organisation and localisation of the FSHD-associated tandem array in primates: implications for the origin and evolution of the 3.3 kb repeat family. Chromosoma 105, 180-189.
Domer, A., and Schultheiss, H. P. (2000). The myocardial expression of the adenine nucleotide translocator isoforms is specifically altered in dilated cardiomyopathy. Herz 25, 176-180.
Dunger, D. B., Ong, K. K., Huxtable, S. J., Sherriff, A., Woods, K. A., Ahmed, M. L., Golding, J., Pembrey, M. E., Ring, S., Bennett, S. T., and Todd, J. A. (1998). Association of the INS VNTR with size at birth. ALSPAC study team. Avon longitudinal study of pregnancy and childhood. Nat. Genet. 19, 98-100.
Eng, J. K., McCormack, A. L., and Yates, J. R., Iii. (1994). An approach to correlate tandem mass spectral data of peptides with amino acid sequences in a protein database. J. Am. Soc. Mass Spectrom. 5, 976-989.
Fisher, J., and Upadhyaya, M. (1997). Molecular genetics of facio and scapulohumeral muscular dystrophy (FSHD). Neuromuscul. Disord. 7, 55-62.
Gabriels, J., Beckers, M. C., Ding, H., De Vriese, A., Plaisance, S., van der Maarel, S. M., Padberg, G. W., Frants, R. R., Hewitt, J. E., Collen, D., and Belayew, A. (1999). Nucleotide sequence of the partially deleted D4Z4 locus in a patient with FSHD identifies a putative gene within each 3.3 kb element. Gene 236, 25-32.
Hanakahi, L. A., Dempsey, L. A., Li, M. J., and Maizels, N. (1997). Nucleolin is one component of theB cell-specific transcription factor and switch region binding protein, LR1. Proc. Natl. Acad. Sci. USA 94, 3605-3610.
Hewitt, J. E., Lyle, R., Clark, L. N., Valleley, E. M., Wright, T. J., Wijmenga, C., van Deutekom, J. C., Francis, F., Sharpe, P. T., Hofker, M., et al. (1994). Analysis of the tandem repeat locus D4Z4 associated with facioscapulohumeral muscular dystrophy. Hum. Mol. Genet. 3, 1287-1295.
Hsu, Y. D., Kao, M. C., Shyu, W. C., Lin, J. C., Huang, N. E., Sun, H. F., Yang, K. D., and Tsao, W. L. (1997). Application of chromosome 4q35-qter marker (pFR-1) for DNA rearrangement of facioscapulohumeral muscular dystrophy patients in Taiwan. J. Neurol. Sci. 149, 73-79.
Kaukonen, J., Juselius, J. K., Tiranti, V., Kyttala, A., Zeviani, M., Comi, G. P., Keranen, S., Peltonen, L., and Suomalainen, A. (2000). Role of adenine nucleotide translocator 1 in mtDNA maintenance. Science 289, 782-785.
Kennedy, G. C., German, M. S., and Rutter, W. J. (1995). The minisatellite in the diabetes susceptibility locus IDDM2 regulates insulin transcription. Nat. Genet. 9, 293-298.
Krontiris, T. G., Devlin, B., Karp, D. D., Robert, N. J., and Risch, N. (1993). An association between the risk of cancer and mutations in the HRAS1 minisatellite locus. N. Engl. J. Med. 329, 517-523.
Lehming, N., Le Saux, A., Schuller, J., and Ptashne, M. (1998). Chromatin components as part of a putative transcriptional repressing complex. Proc. Natl. Acad. Sci. USA 95, 7322-7326.
Lunt, P. W. (1998). 44th ENMC international workshop: facioscapulohumeral muscular dystrophy: molecular studies 19-21 July 1996, Naarden, The Netherlands. Neuromuscul. Disord. 8, 126-130.
Lunt, P. W., Jardine, P. E., Koch, M. C., Maynard, J., Osborn, M., Williams, M., Harper, P. S., and Upadhyaya, M. (1995). Correlation between fragment size at D4F104S1 and age at onset or at wheelchair use, with a possible generational effect, accounts for much phenotypic variation in 4q35-facioscapulohumeral muscular dystrophy (FSHD). Hum. Mol. Genet. 4, 951-958.
Lustig, A. J. (1998). Mechanisms of silencing in Saccharomyces cerevisiae. Curr. Opin. Genet. Dev. 8, 233-239.
Lyle, R., Wright, T. J., Clark, L. N., and Hewitt, J. E. (1995). The FSHD associated repeat, D4Z4, is a member of a dispersed family of homeobox-containing repeats, subsets of which are clustered on the short arms of the acrocentric chromosomes. Genomics 28, 389-397.
Morcos, P. A. (2001). Achieving efficient delivery of morpholino oligos in cultured cells. Genesis 30, 94-102.
Napoli, L., Bordoni, A., Zeviani, M., Hadjigeorgiou, G. M., Sciacco, M., Tiranti, V., Terentiou, A., Moggio, M., Papadimitriou, A., Scarlato, G., and Comi, G. P. (2001). A novel missense adenine nucleotide translocator-1 gene mutation in a Greek adPEO family. Neurology 57, 2295-2308.
Orlando, V., and Paro, R. (1993). Mapping Polycomb-repressed domains in the bithorax complex using in vivo formaldehyde cross-linked chromatin. Cell 75, 1187-1198.
Padberg, G. W. (1982). Facioscapulohumeral disease. MD Thesis, Leiden University, Leiden, The Netherlands.
Pryde, F. E., and Louis, E. J. (1999). Limitations of silencing at native yeast telomeres. EMBO J. 18, 2538-2550. chromatin structure in the pathogenesis of the disease.
Renauld, H., Aparicio, O. M., Zierath, P. D., Billington, B. L., Chhablani, S. K., and Gottschling, D. E. (1993). Silent domains are assembled continuously from the telomere and are defined by promoter distance and by SIR3 dosage. Genes Dev. 7, 1133-1145. transcription factor YY1 and stimulates YY1 DNA binding. J. Cell.
Ricci, E., Galluzzi, G., Deidda, G., Cacurri, S., Colantoni, L., Merico, B., Piazzo, N., Servidei, S., Vigneti, E., Pasceri, V., et al. (1999). Progress in the molecular diagnosis of facioscapulohumeral muscular dystrophy and correlation between the number of KpnI repeats at the 4q35 locus and clinical phenotype. Ann. Neurol. 45, 751-757.
Sandri, M., El Meslemani, A. H., Sandri, C., Schjerling, P., Vissing, K., Andersen, J. L., Rossini, K., Carraro, U., and Angelini, C. (2001). Caspase 3 expression correlates with skeletal muscle apoptosis in Duchenne and facioscapulo human muscular dystrophy. A potential target for pharmacological treatment? J. Neuropathol. Exp. Neurol. 60, 302-312.
Sarfarazi, M., Wijmenga, C., Upadhyaya, M., Weiffenbach, B., Hyser, C., Mathews, K., Murray, J., Gilbert, J., Pericak-Vance, M., Lunt, P., et al. (1992). Regional mapping of facioscapulohumeral muscular dystrophy gene on 4q35: combined analysis of an international consortium. Am. J. Hum. Genet. 51, 396-403.
Schulz, M., Schneider, S., Lottspeich, F., Renkawitz, R., and Eggert, M. (2001). Identification of nucleolin as a glucocorticoid receptor interacting protein. Biochem. Biophys. Res. Commun. 280, 476-480.
Tawil, R., Forrester, J., Griggs, R. C., Mendell, J., Kissel, J., McDermott, M., King, W., Weiffenbach, B., and Figlewicz, D. (1996). Evidence for anticipation and association of deletion size with severity in facioscapulohumeral muscular dystrophy. The FSH-DY Group. Ann. Neurol. 39, 744-748.
Tews, D. S., and Goebel, H. H. (1997). DNA-fragmentation and expression of apoptosis-related proteins in muscular dystrophies. Neuropathol. Appl. Neurobiol. 23, 331-338.
Thomas, M. J., and Seto, E. (1999). Unlocking the mechanisms of transcription factor YY1: are chromatin modifying enzymes the key? Gene 236, 197-208.
Thomas, J. O., and Travers, A. A. (2001). HMG1 and 2, and related ‘architectural’ DNA-binding proteins. Trends Biochem. Sci. 3, 167-174.
Tupler, R., Berardinelli, A., Barbierato, L., Frants, R., Hewitt, J. E.,
Lanzi, G., Maraschio, P., and Tiepolo, L. (1996). Monosomy of distal 4q does not cause facioscapulohumeral muscular dystrophy. J. Med. Genet. 33, 366-370.
Tupler, R., Perini, G., Pellegrino, M. A., and Green, M. R. (1999). Profound misregulation of muscle-specific gene expression in facioscapulohumeral muscular dystrophy. Proc. Natl. Acad. Sci. USA 96, 12650-12654.
van Deutekom, J. C., Lemmers, R. J., Grewal, P. K., van Geel, M., Romberg, S., Dauwerse, H. G., Wright, T. J., Padberg, G. W., Hofker, M. H., Hewitt, J. E., and Frants, R. R. (1996). Identification of the first gene (FRG1) from the FSHD region on human chromosome 4q35. Hum. Mol. Genet. 5, 581-590.
van Geel, M., Heather, L. J., Lyle, R., Hewitt, J. E., Frants, R. R., and de Jong, P. J. (1999). The FSHD region on human chromosome 4q35 contains potential coding regions among pseudogenes and a high density of repeat elements. Genomics 61, 55-65.
van Geel, M., van Deutekom, J. C., van Staalduinen, A., Lemmers, R. J., Dickson, M. C., Hofker, M. H., Padberg, G. W., Hewitt, J. E., de Jong, P. J., and Frants, R. R. (2000). Identification of a novel beta-tubulin subfamily with one member (TUBB4Q) located near the telomere of chromosome region 4q35. Cytogenet. Cell Genet. 88, 316-321.
Wijmenga, C., Hewitt, J. E., Sandkuijl, L. A., Clark, L. N., Wright, T. J., Dauwerse, H. G., Gruter, A. M., Hofker, M. H., Moerer, P., Williamson, R., et al. (1992). Chromosome 4q DNA rearrangements associated with facioscapulohumeral muscular dystrophy. Nat. Genet. 2, 26-30.
Winokur, S. T., Bengtsson, U., Feddersen, J., Mathews, K. D., Weiffenbach, B., Bailey, H., Markovich, R. P., Murray, J. C., Wasmuth, J. J., Altherr, M. R., et al. (1994). The DNA rearrangement associated J. J., Altherr, M. R., et al. (1994). The DNA rearrangement associated with facioscapulohumeral muscular dystrophy involves a hetero-chromatin-associated repetitive element: implications for a role of chromatin structure in the pathogenesis of the disease. Chromosome Res. 2, 225-234.
Xie, J., Briggs, J. A., and Briggs, R. C. (1998). Human hematopoieticcell specific nuclear protein MNDA interacts with the multifunctional transcription factor YY1 and stimulates YY1 DNA binding. J. Cell. Biochem. 70, 489-506.
Yant, S. R., Zhu, W., Millinoff, D., Slightom, J. L., Goodman, M., and Gumucio, D. L. (1995). High affinity YY1 binding motifs: identification of two core types (ACAT and CCAT) and distribution of potential of two core types (ACAT and CCAT) and distribution of potential Res. 23, 4353-4362.
Ying, G. G., Proost, P., van Damme, J., Bruschi, M., Introna, M., and Golay, J. (2000). Nucleolin, a novel partner for the Myb transcription factor family that regulates their activity. J. Biol. Chem. 275, 4152-4158.
Zatz, M., Marie, S. K., Passos-Bueno, M. R., Vainzof, M., Campiotto, S., Cerqueira, A., Wijmenga, C., Padberg, G., and Frants, R. (1995). High proportion of new mutations and possible anticipation in Brazilian facioscapulohumeral muscular dystrophy families. Am. J. Hum. Genet. 56, 99-105.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying a candidate therapeutic compound for treating FSHD, the method comprising,
(a) providing a D4Z4 binding element;
(b) contacting the D4Z4 binding element with a test compound; and
(c) determining whether the test compound interacts with the D4Z4 binding element,
wherein an interaction between the D4Z4 binding element and the test compound indicates that the test compound is a candidate therapeutic compound.

2. The method of claim 1, wherein the D4Z4 binding element is in a cell that expresses a 4q35 gene.

3. The method of claim 2, further comprising the step of determining the level of expression of a 4q35 gene compared to a reference.

4. The method of claim 3, wherein the 4q35 gene is FSHD region gene 1 (FRG1), FSHD region gene 2 (FRG2), or adenine nucleotide translocator-1 gene (ANT1).

5. The method of claim 1, wherein the cell is a muscle cell.

6. The method of claim 5, wherein the cell is from a subject that has FSHD.

7. The method of claim 1, wherein the interaction is the binding of the test compound to the D4Z4 binding element.

8. A method of identifying a candidate compound for treating FSHD, the method comprising,
(a) providing a cell that can express a D4Z4 recognition complex component;
(b) contacting the cell with a test compound; and
(c) measuring expression of the D4Z4 recognition complex component,
wherein an increase in expression of the D4Z4 recognition complex component compared to a reference cell that was not contacted with the test compound indicates that the test compound is a candidate compound for treating FSHD.

8. The method of claim 8, wherein the D4Z4 recognition complex component is YY1, HMGB2, or nucleolin.

9. A method of identifying a candidate compound for treating FSHD, the method comprising,
(a) providing a D4Z4 binding element (DBE) and a D4Z4 recognition complex (DRC) under conditions such that the DBE and the DRC can interact;
(b) contacting the D4Z4 binding element and DRC or a DRC component with a test compound; and
(c) determining whether the test compound affects the interaction between the D4Z4 binding element and the DRC or DRC component,
wherein an increase in the interaction between the D4Z4 binding element and the DRC or DRC component in the presence of the test compound indicates that the test compound is a candidate compound.

10. The method of claim 9, wherein the DRC component is YY1, HMGB2, or nucleolin.

11. A method of determining whether a treatment for FSHD is effective, the method comprising
(a) obtaining a biological sample from a subject being treated for FSHD,
(b) determining the level of expression of a 4q35 gene,
(c) comparing the level of expression of the 4q35 gene to a reference, wherein a decrease in the level of expression of the FSHD gene relative to a reference indicates that the FSHD treatment is effective.

12. The method of claim 11, wherein the 4q35 gene is FSHD region gene 1 (FRG1), FSHD region gene 2 (FRG2), or adenine nucleotide translocator-1 gene (ANT1).

13. The method of claim 11, wherein expression of at least two 4q35 genes is decreased in the biological sample.

14. A method of treating a subject having or at risk for FSHD, the method comprising administering to the subject a compound that increases the expression or activity of at least one component of a D4Z4 recognition complex (DRC).

15. The method of claim 14, wherein the component of the D4Z4 recognition complex is YY1, HMGB2, or nucleolin.

16. A method of identifying a subject having or at risk of having facioscapulohumeral muscular dystrophy (FSHD), the method comprising
(a) obtaining a biological sample from the subject
(b) determining the level of expression of a 4q35 gene in the sample
(c) comparing the level of expression of the 4q35 gene in the sample compared to a control obtained from an individual that does not have FSHD, wherein increased expression of the 4q35 gene indicates that the subject is at risk of having or has FSHD.

17. The method of claim 16, wherein the 4q35 gene is FSHD region gene 1 (FRG1), FSHD region gene 2 (FRG2), or adenine nucleotide translocator-1 gene (ANT1).

18. The method of claim 16, wherein the biological sample is from muscle.

19. A non-human transgenic animal one or more of whose cells can overexpress at least one 4q35 gene selected from the group consisting of FSHD region gene 1 (FRG1), FSHD region gene 2 (FRG2), or adenine nucleotide translocator-1 gene (ANT1), wherein the overexpression of the transgene results in the animal exhibiting symptoms of FSHD.

20. The non-human transgenic animal of claim 19, wherein the 4q35 gene is FRG1.

21. A method of determining whether a treatment for FSHD is effective, the method comprising
(a) obtaining a non-human transgenic animal model of FSHD overexpressing a 4q35 gene;
(b) administering the treatment to the animal; and
(c) evaluating the effect of the treatment on the animal,
wherein an improvement in a symptom of FSHD indicates that the FSHD treatment is effective.
